SEQUENCE LISTING (135 sequences)

SEQ ID NO: 1 - DNA, 18 bp, synthetic construct
  source 1..18: mol_type = other DNA
cagaagtccctgttgcta

SEQ ID NO: 2 - DNA, 21 bp, synthetic construct
  source 1..21: mol_type = other DNA
cgaggagaattagtctgagtc

SEQ ID NO: 3 - DNA, 47 bp, synthetic construct
  source 1..47: mol_type = other DNA
tgcacgtgtttgaaaaacattagaattcatgcagatcaacttactcc

SEQ ID NO: 4 - DNA, 45 bp, synthetic construct
  source 1..45: mol_type = other DNA
ggctgtttaataggggctgaacatgataactagcgcatatacctg

SEQ ID NO: 5 - DNA, 24 bp, synthetic construct
  source 1..24: mol_type = other DNA
cctgtagaataaacacgccaagta

SEQ ID NO: 6 - DNA, 22 bp, synthetic construct
  source 1..22: mol_type = other DNA
ctcatatgagtgtgacataccc

SEQ ID NO: 7 - DNA, 23 bp, synthetic construct
  source 1..23: mol_type = other DNA
ccaatttaatagtgctattggca

SEQ ID NO: 8 - DNA, 18 bp, synthetic construct
  source 1..18: mol_type = other DNA
gcacttcagcctcaactt

SEQ ID NO: 9 - DNA, 47 bp, synthetic construct
  source 1..47: mol_type = other DNA
gcattttggttgaccacatcttgaaaattcaagactcactttcttcc

SEQ ID NO: 10 - DNA, 44 bp, synthetic construct
  source 1..44: mol_type = other DNA
gctttaaacacgcttgttaaacaactgtcaagacgtgaaaggat

SEQ ID NO: 11 - DNA, 19 bp, synthetic construct
  source 1..19: mol_type = other DNA
ttttccaagtgcacttgct

SEQ ID NO: 12 - DNA, 20 bp, synthetic construct
  source 1..20: mol_type = other DNA
agctccaattttggtgcaat

SEQ ID NO: 13 - DNA, 18 bp, synthetic construct
  source 1..18: mol_type = other DNA
ttggtgcaggtatatgcg

SEQ ID NO: 14 - DNA, 23 bp, synthetic construct
  source 1..23: mol_type = other DNA
acattgtacaatctactgatgtc

SEQ ID NO: 15 - DNA, 49 bp, synthetic construct
  source 1..49: mol_type = other DNA
aggcaatgatggattgactagctacttatcagactcagactaattctcc

SEQ ID NO: 16 - DNA, 45 bp, synthetic construct
  source 1..45: mol_type = other DNA
taactctattgccatacccacaaatttggtcatagacactggtag

SEQ ID NO: 17 - DNA, 20 bp, synthetic construct
  source 1..20: mol_type = other DNA
tagtgttaccacagaaattc

SEQ ID NO: 18 - DNA, 43 bp, synthetic construct
  source 1..43: mol_type = other DNA
ctaataactctattgccatacccttggtcatagacactggtag

SEQ ID NO: 19 - DNA, 18 bp, synthetic construct
  source 1..18: mol_type = other DNA
attggtgcaggtatatgc

SEQ ID NO: 20 - DNA, 21 bp, synthetic construct
  source 1..21: mol_type = other DNA
tggtagaatttctgtggtaac

SEQ ID NO: 21 - DNA, 44 bp, synthetic construct
  source 1..44: mol_type = other DNA
taggcaatgatggattgactagctcagactcagactaattctcc

SEQ ID NO: 22 - DNA, 43 bp, synthetic construct
  source 1..43: mol_type = other DNA
tgtaggcaatgatggattgactatcagactcagactaattctc

SEQ ID NO: 23 - DNA, 45 bp, synthetic construct
  source 1..45: mol_type = other DNA
cactatgtcacttggtgcagctaatagtaaaatttgtgggtatgg

SEQ ID NO: 24 - DNA, 19 bp, synthetic construct
  source 1..19: mol_type = other DNA
tacactacgtgcccgccga

SEQ ID NO: 25 - DNA, 18 bp, synthetic construct
  source 1..18: mol_type = other DNA
aggctgtttaataggggc

SEQ ID NO: 26 - DNA, 19 bp, synthetic construct
  source 1..19: mol_type = other DNA
atttgtgggtatggcaata

SEQ ID NO: 27 - DNA, 44 bp, synthetic construct
  source 1..44: mol_type = other DNA
tgagtctgataactagcgcatatatcaacaactcatatgagtgt

SEQ ID NO: 28 - DNA, 39 bp, synthetic construct
  source 1..39: mol_type = other DNA
ggcacgtagtgtagctagtcaagcaactgaattttctgc

SEQ ID NO: 29 - DNA, 20 bp, synthetic construct
  source 1..20: mol_type = other DNA
cctgcaccaatgggtatgtc

SEQ ID NO: 30 - DNA, 21 bp, synthetic construct
  source 1..21: mol_type = other DNA
tgcctacactatgtcacttgg

SEQ ID NO: 31 - DNA, 22 bp, synthetic construct
  source 1..22: mol_type = other DNA
acaatcttgattctaaggttgg

SEQ ID NO: 32 - DNA, 21 bp, synthetic construct
  source 1..21: mol_type = other DNA
actactactctgtatggttgg

SEQ ID NO: 33 - DNA, 50 bp, synthetic construct
  source 1..50: mol_type = other DNA
cggcctgatagatttcagttgaaatttacctgtatagattgtttaggaag

SEQ ID NO: 34 - DNA, 43 bp, synthetic construct
  source 1..43: mol_type = other DNA
gtagcacaccttgtaatggtgttgcaacaccattagtgggttg

SEQ ID NO: 35 - DNA, 24 bp, synthetic construct
  source 1..24: mol_type = other DNA
ctctctcaaaaggtttgagattag

SEQ ID NO: 36 - DNA, 25 bp, synthetic construct
  source 1..25: mol_type = other DNA
gttactttcctttacaatcatatgg

SEQ ID NO: 37 - DNA, 25 bp, synthetic construct
  source 1..25: mol_type = other DNA
cggcctgatagatttcagttgaaat

SEQ ID NO: 38 - DNA, 20 bp, synthetic construct
  source 1..20: mol_type = other DNA
ctcaaaccttttgagagaga

SEQ ID NO: 39 - DNA, 18 bp, synthetic construct
  source 1..18: mol_type = other DNA
ggtgcatgtagaagttca

SEQ ID NO: 40 - DNA, 21 bp, synthetic construct
  source 1..21: mol_type = other DNA
tccacaaacagttgctggtgc

SEQ ID NO: 41 - DNA, 23 bp, synthetic construct
  source 1..23: mol_type = other DNA
ccacaaacagttgctggtgcatg

SEQ ID NO: 42 - DNA, 48 bp, synthetic construct
  source 1..48: mol_type = other DNA
gtaacaattaaaaccttcaacaccatatttcaactgaaatctatcagg

SEQ ID NO: 43 - DNA, 45 bp, synthetic construct
  source 1..45: mol_type = other DNA
ggaaagtaacaattaaaacctatatttcaactgaaatctatcagg

SEQ ID NO: 44 - DNA, 47 bp, synthetic construct
  source 1..47: mol_type = other DNA
ttcctttacaatcatatggtttccaaaagaaagtactactactctgt

SEQ ID NO: 45 - DNA, 48 bp, synthetic construct
  source 1..48: mol_type = other DNA
ttacaatcatatggtttccaaccccaaaagaaagtactactactctgt

SEQ ID NO: 46 - DNA, 20 bp, synthetic construct
  source 1..20: mol_type = other DNA
cattacaaggtgtgctaccg

SEQ ID NO: 47 - DNA, 21 bp, synthetic construct
  source 1..21: mol_type = other DNA
cccactaatggtgttggttac

SEQ ID NO: 48 - DNA, 20 bp, synthetic construct
  source 1..20: mol_type = other DNA
aatggtgttggttaccaacc

SEQ ID NO: 49 - DNA, 23 bp, synthetic construct
  source 1..23: mol_type = other DNA
ttcctttacaatcatatggtttc

SEQ ID NO: 50 - DNA, 23 bp, synthetic construct
  source 1..23: mol_type = other DNA
actgagtctaacaaaaagtttct

SEQ ID NO: 51 - DNA, 20 bp, synthetic construct
  source 1..20: mol_type = other DNA
cctgataaagaacagcaacc

SEQ ID NO: 52 - DNA, 40 bp, synthetic construct
  source 1..40: mol_type = other DNA
ctgtggatcacggacagcatcgcctttccaacaatttggc

SEQ ID NO: 53 - DNA, 48 bp, synthetic construct
  source 1..48: mol_type = other DNA
acttgagattcttgacattacaccatggttagaagtatttgttcctgg

SEQ ID NO: 54 - DNA, 21 bp, synthetic construct
  source 1..21: mol_type = other DNA
agtagtgtcagcaatgtctct

SEQ ID NO: 55 - DNA, 20 bp, synthetic construct
  source 1..20: mol_type = other DNA
tgttcttttggtggtgtcag

SEQ ID NO: 56 - DNA, 18 bp, synthetic construct
  source 1..18: mol_type = other DNA
ttctttcacacgtggtgt

SEQ ID NO: 57 - DNA, 21 bp, synthetic construct
  source 1..21: mol_type = other DNA
agtggaagcaaaataaacacc

SEQ ID NO: 58 - DNA, 48 bp, synthetic construct
  source 1..48: mol_type = other DNA
aggtaagaacaagtcctgagttgattattaccctgacaaagttttcag

SEQ ID NO: 59 - DNA, 45 bp, synthetic construct
  source 1..45: mol_type = other DNA
tcttttccaatgttacttggttccagacagggttatcaaacctct

SEQ ID NO: 60 - DNA, 25 bp, synthetic construct
  source 1..25: mol_type = other DNA
gctatacatgtctctgggaccaatg

SEQ ID NO: 61 - DNA, 22 bp, synthetic construct
  source 1..22: mol_type = other DNA
ttcttaggaatcatcacaactg

SEQ ID NO: 62 - DNA, 21 bp, synthetic construct
  source 1..21: mol_type = other DNA
atatcgatgtactgaatgggt

SEQ ID NO: 63 - DNA, 47 bp, synthetic construct
  source 1..47: mol_type = other DNA
gacacgggtcatcaactacatatgcaccaagaatgtagtttacagtc

SEQ ID NO: 64 - DNA, 45 bp, synthetic construct
  source 1..45: mol_type = other DNA
gagtaggagctagaaaatcagcacgatttagaaccagcctcatcc

SEQ ID NO: 65 - DNA, 19 bp, synthetic construct
  source 1..19: mol_type = other DNA
acgcctaaacgaacatgaa

SEQ ID NO: 66 - DNA, 18 bp, synthetic construct
  source 1..18: mol_type = other DNA
agaaccagcctcatccac

SEQ ID NO: 67 - DNA, 49 bp, synthetic construct
  source 1..49: mol_type = other DNA
ggttgatgttgagtacatgactgtacttgttttcttaggaatcatcaca

SEQ ID NO: 68 - DNA, 46 bp, synthetic construct
  source 1..46: mol_type = other DNA
atatgtagttgatgacccgtgtcctaaaggtgctgattttctagct

SEQ ID NO: 69 - DNA, 25 bp, synthetic construct
  source 1..25: mol_type = other DNA
aactacattcttggtgaaatgcagc

SEQ ID NO: 70 - DNA, 23 bp, synthetic construct
  source 1..23: mol_type = other DNA
aactcaatatgagtatggtactg

SEQ ID NO: 71 - DNA, 21 bp, synthetic construct
  source 1..21: mol_type = other DNA
ctgaacaactggtgtaagttc

SEQ ID NO: 72 - DNA, 45 bp, synthetic construct
  source 1..45: mol_type = other DNA
ttgctcttcttcaggttgaagagcttaccaaggtaaacctttgga

SEQ ID NO: 73 - DNA, 42 bp, synthetic construct
  source 1..42: mol_type = other DNA
ttggtcaacaagacggcagtcatctctaattgaggttgaacc

SEQ ID NO: 74 - DNA, 19 bp, synthetic construct
  source 1..19: mol_type = other DNA
agcagaagtggcaccaaat

SEQ ID NO: 75 - DNA, 25 bp, synthetic construct
  source 1..25: mol_type = other DNA
gaggacaatcagacaactactattc

SEQ ID NO: 76 - DNA, 18 bp, synthetic construct
  source 1..18: mol_type = other DNA
tgttatcttgccactgca

SEQ ID NO: 77 - DNA, 23 bp, synthetic construct
  source 1..23: mol_type = other DNA
agaatctaaattggcatgttgaa

SEQ ID NO: 78 - DNA, 48 bp, synthetic construct
  source 1..48: mol_type = other DNA
ccttgctctgtaataagcatcttgtttaacactccaacaaatagagtt

SEQ ID NO: 79 - DNA, 48 bp, synthetic construct
  source 1..48: mol_type = other DNA
tgctctgtaataagcatcttgtagattaacactccaacaaatagagtt

SEQ ID NO: 80 - DNA, 45 bp, synthetic construct
  source 1..45: mol_type = other DNA
ctggtgaagctgctaacttttgttgtttctctaacatcacctaac

SEQ ID NO: 81 - DNA, 45 bp, synthetic construct
  source 1..45: mol_type = other DNA
gtgcacttatcttagcctactgttgtttctctaacatcacctaac

SEQ ID NO: 82 - DNA, 46 bp, synthetic construct
  source 1..46: mol_type = other DNA
acttttgtgcacttatcttagccttgtttctctaacatcacctaac

SEQ ID NO: 83 - DNA, 21 bp, synthetic construct
  source 1..21: mol_type = other DNA
agagcaggtggattaaacttc

SEQ ID NO: 84 - DNA, 24 bp, synthetic construct
  source 1..24: mol_type = other DNA
gcctactgtaataagacagtaggt

SEQ ID NO: 85 - DNA, 21 bp, synthetic construct
  source 1..21: mol_type = other DNA
gcttttgcaatgatgtttgtc

SEQ ID NO: 86 - DNA, 21 bp, synthetic construct
  source 1..21: mol_type = other DNA
gttcttgctgtcataaggatt

SEQ ID NO: 87 - DNA, 50 bp, synthetic construct
  source 1..50: mol_type = other DNA
ccaactagcaggcatatagaccatacatttctctgtttgtttttgttacc

SEQ ID NO: 88 - DNA, 46 bp, synthetic construct
  source 1..46: mol_type = other DNA
gtgatgcgtattatgacatggttggatgcatacataacacagtctt

SEQ ID NO: 89 - DNA, 19 bp, synthetic construct
  source 1..19: mol_type = other DNA
aagctacagtggcaagaga

SEQ ID NO: 90 - DNA, 22 bp, synthetic construct
  source 1..22: mol_type = other DNA
tggttgatactagtttgtctgg

SEQ ID NO: 91 - DNA, 22 bp, synthetic construct
  source 1..22: mol_type = other DNA
cggtaattatacagtttcctgt

SEQ ID NO: 92 - DNA, 19 bp, synthetic construct
  source 1..19: mol_type = other DNA
tgcatttcgctgattttgg

SEQ ID NO: 93 - DNA, 46 bp, synthetic construct
  source 1..46: mol_type = other DNA
gaacgaacaacgcactacaagataccttttacaattaattgccagg

SEQ ID NO: 94 - DNA, 48 bp, synthetic construct
  source 1..48: mol_type = other DNA
gagtatcatgacgttcgtgttgtgtccattatcagacattttagtttg

SEQ ID NO: 95 - DNA, 22 bp, synthetic construct
  source 1..22: mol_type = other DNA
gttgttcgttctatgaagactt

SEQ ID NO: 96 - DNA, 18 bp, synthetic construct
  source 1..18: mol_type = other DNA
ccgacgttgttttgatcg

SEQ ID NO: 97 - DNA, 46 bp, synthetic construct
  source 1..46: mol_type = other DNA
tggggtccattatcagacattttagtagagtatcatgacgttcgtg

SEQ ID NO: 98 - DNA, 40 bp, synthetic construct
  source 1..40: mol_type = other DNA
cgaaatgcaccccgcattacgcgttctccattctggttac

SEQ ID NO: 99 - DNA, 21 bp, synthetic construct
  source 1..21: mol_type = other DNA
gaccctcagattcaactggca

SEQ ID NO: 100 - DNA, 21 bp, synthetic construct
  source 1..21: mol_type = other DNA
tgttatggagtgtctcctact

SEQ ID NO: 101 - DNA, 24 bp, synthetic construct
  source 1..24: mol_type = other DNA
tgagattagacttcctaaacaatc

SEQ ID NO: 102 - DNA, 47 bp, synthetic construct
  source 1..47: mol_type = other DNA
gagcgatttgtctgacttcatcatgctttactaatgtctatgcagat

SEQ ID NO: 103 - DNA, 44 bp, synthetic construct
  source 1..44: mol_type = other DNA
taccagatgattttacaggctgcccaccaaccttagaatcaaga

SEQ ID NO: 104 - DNA, 25 bp, synthetic construct
  source 1..25: mol_type = other DNA
gttatagcttggaattctaacaatc

SEQ ID NO: 105 - DNA, 21 bp, synthetic construct
  source 1..21: mol_type = other DNA
tgtcctatataattccgcatc

SEQ ID NO: 106 - DNA, 23 bp, synthetic construct
  source 1..23: mol_type = other DNA
gaatcaagattgttagaattcca

SEQ ID NO: 107 - DNA, 45 bp, synthetic construct
  source 1..45: mol_type = other DNA
tgcatagacattagtaaagcagagccacttttaagtgttatggag

SEQ ID NO: 108 - DNA, 42 bp, synthetic construct
  source 1..42: mol_type = other DNA
tgatgaagtcagacaaatcgctgcctgtaaaatcatctggta

SEQ ID NO: 109 - DNA, 42 bp, synthetic construct
  source 1..42: mol_type = other DNA
gaggtgatgaagtcagacaaatgcctgtaaaatcatctggta

SEQ ID NO: 110 - DNA, 22 bp, synthetic construct
  source 1..22: mol_type = other DNA
atcatttaatttagtaggagac

SEQ ID NO: 111 - DNA, 15 bp, synthetic construct
  source 1..15: mol_type = other DNA
cagggcaaactggaa

SEQ ID NO: 112 - DNA, 24 bp, synthetic construct
  source 1..24: mol_type = other DNA
tgcatagacattagtaaagcagag

SEQ ID NO: 113 - DNA, 21 bp, synthetic construct
  source 1..21: mol_type = other DNA
atctctgctttactaatgtct

SEQ ID NO: 114 - DNA, 23 bp, synthetic construct
  source 1..23: mol_type = other DNA
gagattagacttcctaaacaatc

SEQ ID NO: 115 - DNA, 43 bp, synthetic construct
  source 1..43: mol_type = other DNA
atctttccagtttgccctgggcagattcatttgtaattagagg

SEQ ID NO: 116 - DNA, 46 bp, synthetic construct
  source 1..46: mol_type = other DNA
taccagatgattttacaggctgcattataattaccaccaaccttag

SEQ ID NO: 117 - DNA, 43 bp, synthetic construct
  source 1..43: mol_type = other DNA
gctccagggcaaactggaaagcagattcatttgtaattagagg

SEQ ID NO: 118 - DNA, 21 bp, synthetic construct
  source 1..21: mol_type = other DNA
gcgatttgtctgacttcatca

SEQ ID NO: 119 - DNA, 22 bp, synthetic construct
  source 1..22: mol_type = other DNA
gcttggaattctaacaatcttg

SEQ ID NO: 120 - DNA, 23 bp, synthetic construct
  source 1..23: mol_type = other DNA
taccagatgattttacaggctgc

SEQ ID NO: 121 - DNA, 23 bp, synthetic construct
  source 1..23: mol_type = other DNA
  modified_base 23: mod_base = OTHER; note = Locked Nucleic Acid
agcacaccttgtaatggtgttaa

SEQ ID NO: 122 - DNA, 23 bp, synthetic construct
  source 1..23: mol_type = other DNA
  modified_base 21: mod_base = OTHER; note = Locked Nucleic Acid
agcacaccttgtaatggtgttaa

SEQ ID NO: 123 - DNA, 23 bp, synthetic construct
  source 1..23: mol_type = other DNA
  modified_base 23: mod_base = OTHER; note = Locked Nucleic Acid
  modified_base 21: mod_base = OTHER; note = Locked Nucleic Acid
agcacaccttgtaatggtgttaa

SEQ ID NO: 124 - DNA, 22 bp, synthetic construct
  source 1..22: mol_type = other DNA
  modified_base 22: mod_base = OTHER; note = Locked Nucleic Acid
gcacaccttgtaatggtgttaa

SEQ ID NO: 125 - DNA, 22 bp, synthetic construct
  source 1..22: mol_type = other DNA
  modified_base 20: mod_base = OTHER; note = Locked Nucleic Acid
gcacaccttgtaatggtgttaa

SEQ ID NO: 126 - DNA, 22 bp, synthetic construct
  source 1..22: mol_type = other DNA
  modified_base 22: mod_base = OTHER; note = Locked Nucleic Acid
  modified_base 20: mod_base = OTHER; note = Locked Nucleic Acid
gcacaccttgtaatggtgttaa

SEQ ID NO: 127 - DNA, 21 bp, synthetic construct
  source 1..21: mol_type = other DNA
  modified_base 21: mod_base = OTHER; note = Locked Nucleic Acid
cacaccttgtaatggtgttaa

SEQ ID NO: 128 - DNA, 21 bp, synthetic construct
  source 1..21: mol_type = other DNA
  modified_base 19: mod_base = OTHER; note = Locked Nucleic Acid
cacaccttgtaatggtgttaa

SEQ ID NO: 129 - DNA, 21 bp, synthetic construct
  source 1..21: mol_type = other DNA
  modified_base 21: mod_base = OTHER; note = Locked Nucleic Acid
  modified_base 19: mod_base = OTHER; note = Locked Nucleic Acid
cacaccttgtaatggtgttaa

SEQ ID NO: 130 - DNA, 19 bp, synthetic construct
  source 1..19: mol_type = other DNA
  modified_base 17: mod_base = OTHER; note = Locked Nucleic Acid
ccagggcaaactggaaata

SEQ ID NO: 131 - DNA, 18 bp, synthetic construct
  source 1..18: mol_type = other DNA
  modified_base 16: mod_base = OTHER; note = Locked Nucleic Acid
cagggcaaactggaaata

SEQ ID NO: 132 - DNA, 17 bp, synthetic construct
  source 1..17: mol_type = other DNA
  modified_base 15: mod_base = OTHER; note = Locked Nucleic Acid
agggcaaactggaaata

SEQ ID NO: 133 - DNA, 19 bp, synthetic construct
  source 1..19: mol_type = other DNA
  modified_base 17: mod_base = OTHER; note = Locked Nucleic Acid
cactccatcgagatttctc

SEQ ID NO: 134 - DNA, 18 bp, synthetic construct
  source 1..18: mol_type = other DNA
  modified_base 16: mod_base = OTHER; note = Locked Nucleic Acid
actccatcgagatttctc

SEQ ID NO: 135 - DNA, 17 bp, synthetic construct
  source 1..17: mol_type = other DNA
  modified_base 15: mod_base = OTHER; note = Locked Nucleic Acid
ctccatcgagatttctc

(19) United States
(12) Patent Application Publication
(10) Pub. No.: US 2025/0122577 A1
(43) Pub. Date: Apr. 17, 2025

(54) A METHOD FOR DETECTION OF POINT MUTATIONS IN TARGET NUCLEIC ACID USING LOOP-MEDIATED ISOTHERMAL AMPLIFICATION

(71) Applicant: NANYANG TECHNOLOGICAL UNIVERSITY, Singapore (SG)

(72) Inventors: Meng How TAN, Singapore (SG); Kean Hean OOI, Singapore (SG)

(73) Assignee: NANYANG TECHNOLOGICAL UNIVERSITY, Singapore (SG)

(21) Appl. No.: 18/703,278

(22) PCT Filed: Oct. 21, 2022
(86) PCT No.: PCT/SG2022/050756

§ 371 Date: Apr. 19, 2024

(30) Foreign Application Priority Data
Oct. 22, 2021 (SG) 10202111729Y

(51) Int. Cl. C12Q 1/6886; C12Q 1/70

(52) U.S. Cl. CPC C12Q 1/6886; C12Q 1/701; C12Q 2600/156; C12Q 2600/16

(57) ABSTRACT

Various embodiments relate generally to the field of nucleic acid amplification and detection, in particular loop-mediated isothermal nucleic acid amplification and the detection of amplicons using designed detection probes. Moreover, various embodiments also relate to methods and kits for determining the presence or quantity of point mutations in a target nucleic acid molecule in a sample using loop-mediated isothermal amplification, which may be used for identifying genetic variants. In one aspect, the detection probe is a single-stranded probe comprising a nucleotide sequence fully complementary to a nucleotide sequence of the probe binding site comprising the point mutation. In another aspect, the detection probe comprising a nucleotide complementary to the point mutation at the penultimate base relative to the 3′ end of the detection probe.

### CROSS-REFERENCE TO RELATED APPLICATION

This application claims the benefit of priority of Singapore patent application Ser. No. 10/202,111729Y filed 22 Oct. 2021, the content of which being hereby incorporated by reference in its entirety for all purposes.

### TECHNICAL FIELD

Various embodiments relate generally to the field of nucleic acid amplification and detection, in particular loop-mediated isothermal nucleic acid amplification and the detection of point mutations, preferably SNPs, in amplicons using designed detection probes. Moreover, various embodiments also relate to methods and kits for determining the presence or quantity of point mutations in a target nucleic acid molecule in a sample using loop mediated isothermal amplification, which may be used for identifying genetic variants.

### BACKGROUND

Since its emergence, COVID-19 continues to wreak havoc on societies and strain healthcare systems worldwide. To date, over 200 million people have been infected, with a death toll exceeding 4 million. Furthermore, prolonged lockdowns and restricted borders are hurting businesses and costing economies billions of dollars. Based on the World Economic Outlook Report from the International Monetary Fund (IMF), the global economy contracted by 3.5% in 2020, with almost every country posting negative growth. Hence, there is a critical need to formulate plans to cope with the coronavirus, as it becomes entrenched in our day-to-day life.

Besides vaccination, a key strategy to limit transmission of the causative virus, SARS-COV-2, is to test extensively, so that infected individuals can self-isolate quickly with follow-up contact tracing carried out expeditiously if required. Over the course of the pandemic, the scientific community has invested sizeable resources to develop various diagnostic capabilities, ranging from traditional quantitative real-time PCR (qRT-PCR) assays, which are highly sensitive but suffer from a slow turnaround time, to antigen rapid tests (ARTs) and breathalyser systems, which give results in minutes but exhibit much lower sensitivity. A common approach is to implement widespread testing with convenient point-of-care (POC) assays and if positive, follow up with another confirmatory test using qRT-PCR.

Mutant viral strains have emerged and are gaining prevalence throughout many parts of the world. So far, there are four major variants-of-concern, namely Alpha (B.1.1.7, first discovered in the United Kingdom), Beta (lineage B.1.351, first discovered in South Africa), Gamma (lineage P.1, first discovered in Brazil), and Delta (lineage B.1.617.2, first discovered in India). These novel viral variants harbour multiple mutations and may exhibit enhanced transmissibility, reduce treatment efficacy, and affect the extent of immunity generated by vaccination or by a prior infection. For example, the N501Y spike mutation increases affinity to the human ACE2 receptor, thereby enhancing viral transmissibilityl, whereas the E484K mutation reduces the effectiveness of vaccines and antibodies used to treat COVID-19²⁻⁶. As of 2022, the Delta variant is the predominant strain of virus in many countries and appears to be especially contagious with even vaccinated individuals harbouring high viral loads despite showing no or minimal outward symptoms. Hence, SARS-COV-2 variants are a game changer and countries worldwide must ramp up their defences against these and other future variants.

Despite the problems posed by SARS-COV-2 variants, there is currently a shortage of POC diagnostic platforms that permit their specific identification. ARTs and breathalyser systems cannot reveal the presence of any mutations in the virus. Although allele-specific real-time PCR assays can distinguish between wildtype and selected variants⁷⁻¹⁰, they require specialized and expensive instrumentation to run and thus must be carried out in dedicated facilities with the necessary equipment and expertise. For POC settings, a set of CRISPR-based diagnostic assays has recently been developed to detect the Alpha, Beta, and Gamma variants using a customized device¹¹. However, each assay still requires 55 minutes to run. Additionally, the tests are not multiplexed in one reaction tube. Instead, the device has been engineered to hold several sample tubes, with a single target being detected in each tube. Consequently, a negative result from a particular test might be due to insufficient material added, since there is no human internal control in the same tube. More generally, it is noted that multiplexing in CRISPR diagnostics is challenging, especially if only one Cas enzyme is utilized.

During the COVID-19 pandemic, reverse transcription loop-mediated isothermal amplification (RT-LAMP) has emerged as a promising platform for rapid detection of SARS-COV-2. The key reasons for its rising prominence as a diagnostic tool are the ready availability of reagents from multiple vendors and its requirement of only simple instrumentation. In brief, the method relies on a set of four core primers, known as inner primers and displacement primers, as well as a DNA polymerase with strong strand displacement activity for amplification. Often, extra primer sets, namely the loop primers, stem primers, or swarm primers, are added to enhance amplification efficiency. Although various RT-LAMP assays for COVID-19 are now available, most of them will merely detect the coronavirus and cannot distinguish between the different circulating variants as the primers are designed to target highly conserved regions of the SARS-COV-2 genome¹²⁻¹⁶. Furthermore, many existing assays use generic DNA-intercalating dyes or pH-sensitive indicators as a means of readout, which are sequence-independent and therefore do not allow one to discriminate between the different mutant viruses either.

Nevertheless, LAMP can be adapted to detect mutations or single nucleotide polymorphisms (SNPs). The published approaches may be broadly classified into two groups. In the first group, researchers rely on the LAMP primers themselves to discriminate between wildtype and mutant sequences^{15,16}. Here, perfectly matched templates can be amplified much more efficiently than mismatched templates. This is akin to allele-specific PCR assays, where the primers must be empirically tested to ensure that they are sensitive to mismatches. A few variations to such primer-based approaches have also been reported. For example, competitive oligonucleotides that are perfectly complementary to the wildtype sequence can be added in some assays to further prevent or at least delay amplification of the wildtype template^{17,18}. In another variation, the 3′ end of the discriminatory LAMP primer is blocked with the last nucleotide being an RNA residue. Hence, in the presence of a perfectly matched template and RNase H, the blocker will be cleaved off so that strand extension from the primer can take place¹⁹. Alternatively, instead of having a blocked 3′ end, the discriminatory primer can be labelled with a fluorophore and quencher at opposite sides of an RNA residue so that upon RNase H-mediated cleavage, the fluorophore will be separated from the quencher, leading to the generation of a fluorescence signal^{20,21}. Another approach that relies on nuclease activity as well is LEC-LAMP, whereby a loop primer is modified at its 5′ end to incorporate an abasic site flanked by a fluorophore and a quencher²². Thus, an endonuclease IV enzyme can be added into the reaction mix to efficiently cleave the abasic site when the probe hybridizes perfectly with its intended substrate. In contrast, when a mismatch is present near the abasic site, endonuclease activity is greatly reduced. Regardless of the exact method, a major disadvantage of all primer-based approaches is that the design of LAMP primers will be constrained by the location of the mutation or SNP.

In the second group, researchers built into their assays a mutation or SNP detection probe that is independent of the LAMP primers. A key advantage of probe-based approaches is that they provide an extra specificity check to distinguish between bona fide amplicons and spurious products. Various sequence-dependent probes can be utilized. For example, in the universal quenching probe (QProbe) method, a melting curve analysis is performed after amplification²³. A mismatch between the target substrate and the joint DNA will reduce duplex stability, lowering the melting temperature. In the one-step strand displacement (OSD) method, target binding to the toehold occurs less readily for a mismatched substrate, thereby hampering the subsequent strand exchange reaction²⁴. One popular probe that is deployed in LAMP reactions to guard against spurious products is the molecular beacon, which is an oligonucleotide attached with a fluorophore and quencher at opposite ends and designed to fold back on itself to form a hairpin structure^{25,26}. By carefully positioning the location of the genetic variant within the probe, different research groups have successfully utilized molecular beacons to detect mutations, including those found in the SARS-COV-2 genome²⁷. Moreover, to enhance the signal, the molecular beacon can be modified to include some RNA residues, so that upon target recognition, an RNase H enzyme in the reaction can cleave the probe to permanently separate the fluorophore and the quencher²⁸.

Despite their promise, existing probes suffer from multiple shortcomings that hamper their widespread adoption. For example, drawbacks of the QProbe method include the need for a thermal cycler to perform melting curve analysis and the requirement of a guanine at the target site. In addition, design of strand displacement probes can be tricky as binding enthalpies must be carefully balanced to ensure successful strand exchange only for the perfectly matched template but not for the mismatched template. A similar situation arises during the design of molecular beacons. Intermolecular target hybridization should be favoured over the probe's intramolecular interaction only for the perfectly matched substrate but not for the mismatched template and this exquisite balance can be difficult to achieve with point mutations and SNPs.

Therefore, there is still need in the art for an alternative sequence-specific detection method to address the drawbacks of existing approaches. In particular, there is a need in the art for rapid, sensitive, affordable, and easy-to-use assay methods that can be readily programmed to identify different genetic variants including point mutations or SNPs, especially in relation to identifying viral variants as they surface in the community for assisting in diagnostic application.

### SUMMARY

In a first aspect, there is provided a method for determining the presence or quantity of a point mutation, preferably single nucleotide polymorphism (SNP), in a target nucleic acid molecule in a sample using loop mediated isothermal amplification (LAMP), the method comprising:
(a) combining a LAMP reaction mixture, a DNA polymerase with 5′→3′ polymerase activity, and a detection probe with the sample (suspected of containing the target nucleic acid molecule),
   wherein the LAMP reaction mixture comprises a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule,
   wherein the detection probe recognises a probe binding site within target amplicons,
   wherein the detection probe is a single-stranded probe comprising:
      a nucleotide sequence fully complementary to a nucleotide sequence of the probe binding site comprising the point mutation;
      a nucleotide complementary to the point mutation at the penultimate base relative to the 3′ end of the detection probe; and
      a locked nucleic acid (LNA) or peptide nucleic acid (PNA) residue at the first, second, third and/or fourth, preferably third, or second and third, or first and third, or third and fourth nucleotide position relative to the 3′ end of the detection probe,
   wherein the detection probe comprises a quencher-fluorophore pair at opposite ends of the probe at a distance that allow the quencher to quench the fluorophore signal,
   wherein the detection probe can hybridize to said target amplicons under LAMP assay conditions and form a double-stranded probe: target complex,
(b) amplifying the target nucleic acid molecule by LAMP under suitable assay conditions that allow:
   i. generation of the target amplicons;
   ii. hybridization of the detection probe to the target amplicons to form the probe: target complex; and
   iii. cleavage of the detection probe by the DNA polymerase to release the quencher or fluorophore; and
(c) detecting and optionally quantifying the released quencher or fluorophore to determine the presence or quantity of the point mutation in the target nucleic acid molecule in the sample.

In various embodiments, the DNA polymerase with 5′→3′ polymerase activity is a Bst polymerase selected from Bst3 polymerase, Bst2 polymerase and IsoPol+.

In various embodiments, the primer set further comprises two swarm primers.

In various embodiments, the primers are designed to amplify loci in the target nucleic acid molecule comprising the point mutation relative to a reference wild-type nucleotide sequence.

In various embodiments, the nucleotide sequence of the probe binding site comprises the point mutation.

In various embodiments, the LNA or PNA residue is positioned at the third nucleotide position relative to the 3′ end of the detection probe.

In various embodiments, the LNA or PNA residue is positioned at the first and third or second and third nucleotide position relative to the 3′ end of the detection probe.

In various embodiments, the probe binding site is different from and non-overlapping with any one of the primer binding sites.

In various embodiments, the detection probe is 14-23 nucleotide bases in length, preferably 14-21 nucleotide bases in length.

In various embodiments, the detection probe comprises a phosphorothioate bond at the 3′-end.

In various embodiments, the detection probe comprises at least one additional modified nucleotide residue to either increase the detection probes melting temperature (Tm) or binding affinity.

In various embodiments, the fully complementary double-stranded probe: target complex has a higher melting temperature (Tm) in comparison to a non-fully complementary double-stranded probe: target complex in which the target nucleic acid comprises at least one mismatched nucleotide.

In various embodiments, the melting temperature (Tm) of the fully complementary double-stranded probe: target complex is higher by about 1.5° C. or greater in comparison to the non-fully complementary double-stranded probe: target complex.

In various embodiments, the fluorophore is attached to the 5′ end of the detection probe and the quencher is attached to the 3′ end of the detection.

In various embodiments, the method is a multiplexing method and is for determining the presence, or optionally quantity, of a point mutation in two or more target nucleic acid molecules in the sample, wherein the method uses one or more primer sets and one or more detection probes for each target nucleic acid molecule or for multiple related target nucleic acid molecules.

In various embodiments, the point mutation is a biomarker for a disease or condition in a subject, optionally cancer.

In various embodiments, the target nucleic acid molecule is a nucleic acid of a pathogen, optionally a human pathogen, preferably a bacterial, fungal, parasite or viral nucleic acid molecule or a cDNA reverse transcript of a bacterial, fungal, parasite or viral RNA.

In various embodiments, the pathogen is a coronavirus, influenza virus, paramyxovirus or enterovirus.

In various embodiments, the target nucleic acid molecule is a nucleic acid of SARS-COV-2 virus, optionally variants of the SARS-COV-2 virus selected from alpha, beta, gamma and delta variants.

In various embodiments, the nucleic acid of SARS-COV-2 virus is an S-gene comprising a point mutation relative to a reference wild-type S-gene nucleotide sequence.

In various embodiments, the sample is not subjected to a purification step prior to step (a).

In another aspect, there is provided a use of the detection probe as defined herein for determining the presence or quantity of a point mutation in target nucleic acid molecule in a sample using a loop-mediated isothermal amplification method.

In another aspect, there is provided a kit for determining the presence, or quantity, of a point mutation, preferably single nucleotide polymorphism (SNP), in a target nucleic acid molecule in a sample using loop mediated isothermal amplification (LAMP), the kit comprising: a LAMP reaction mixture comprises a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule; a DNA polymerase with 5′→3′ polymerase activity; and a detection probe that recognises a probe binding site within target amplicons, wherein the detection probe is a single-stranded probe comprising: a nucleotide sequence fully complementary to a nucleotide sequence of the probe binding site of the target nucleic acid comprising the point mutation; a nucleotide complementary to the point mutation at the penultimate base relative to the 3′ end of the detection probe; and a locked nucleic acid (LNA) or peptide nucleic acid (PNA) residue at the first, second, third and/or fourth, preferably third, or second and third, or first and third, or third and fourth nucleotide position relative to the 3′ end of the detection probe, wherein the detection probe comprises a quencher-fluorophore pair at opposite ends of the probe at a distance that allows the quencher to quench the fluorophore signal, wherein the detection probe can hybridize to said target amplicons under LAMP assay conditions and form a double-stranded probe: target complex.

### Definitions

The following words and terms used herein shall have the meaning indicated:

Unless specified otherwise, the terms “comprising” and “comprise”, and grammatical variants thereof, are intended to represent “open” or “inclusive” language such that they include recited elements but also permit inclusion of additional, unrecited elements.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail.

As used herein, the term “SNIPER” refers to “Single Nucleotide Polymorphism Effective Reporter” and is a descriptive acronym of the detection probe and method according to various embodiments described herein developed by the inventors of the application. Accordingly, the term “SNIPER method” or “SNIPER assay” refers to the method according to various embodiments described herein. Moreover, the term “SNIPER probe” refers to the detection probe according to various embodiments described herein used in said method and developed by the inventors of the application.

As used herein, the term “LANTERN” refers to “Luminescence from Anticipated Target due to Exonuclease Removal of Nucleotide mismatch” and is a descriptive acronym of an assay and detection probe subject to a published journal³⁰. Briefly, the LANTERN detection probe may be used in isothermal amplification methods and comprises at least one 3′ end nucleotide mismatch in relation to the sequence of the LANTERN probe binding site, such that the LANTERN detection probe hybridizes to the probe binding site in target amplicons except for the 3′ end nucleotide mismatch to form a double-stranded probe: target complex. Essentially, the LANTERN probe does not bind to its target sequence with perfect complementarity and is not fully complementary to the respective target sequence of its probe binding site over the entire length. Accordingly, the term “LANTERN probe” refers to a detection probe as characterised above and detailed in the Ooi et al³⁰, it follows that the term “LANTERN method” or “LANTERN assay” refers to an isothermal amplification method, such as LAMP, that employs the use the LANTERN probe for nucleic acid target detection.

As used herein, the term “at least one”, as used herein, means one or more, for example, 2, 3, 4, 5, 6, 7, 8, 9 or more. If used in relation to a component or agent, the term does not relate to the total number of molecules of the respective component or agent but rather to the number of different species of said component or agent that fall within the definition of broader term.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about”, in the context of concentrations or amounts of components, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

### BRIEF DESCRIPTION OF THE DRAWINGS

Various embodiments will be better understood with reference to the detailed description when considered in conjunction with the non-limiting examples and the accompanying drawings.

FIG. 1 shows the disadvantages of existing probe technologies: (a) RT-LAMP reactions were carried out with RNA templates from S-gene fragments of SARS-COV-2 with or without addition of 2.5U thermostable RNase H2 in a 25 μl reaction; (b) Evaluation of a LANTERN probe targeting the A1708D mutation in the ORF1ab gene of SARS-COV-2. 2E4 copies of in vitro transcribed RNA templates corresponding to A1708 or D1708 were used as template. 0.5U of Q5 High-Fidelity DNA Polymerase was also added in each reaction. Fluorescence measurements here were taken after 30 minutes of RT-LAMP; (c) Evaluation of a LANTERN probe targeting the E484K mutation in the S-gene of SARS-COV-2. RT-LAMP reactions were performed with or without swarm primers. 2E4 copies of in vitro transcribed RNA templates corresponding to E484 or K484 were used as template. 0.5U of Q5 High-Fidelity DNA Polymerase was also added in each reaction. Fluorescence measurements here were taken after 30 minutes of RT-LAMP; and (d) Assessing the effect of additional reverse transcriptase or Bst2 enzyme. In vitro transcribed S-gene templates (corresponding to E484 or K484) were spiked into 0.25 ng of PC9 RNA. 0.5 μM K484-targeting LANTERN probe was used with a S-gene LAMP primer set that excluded the swarm primers. 0.5U of Q5 High-Fidelity DNA Polymerase was also added in each reaction. Fluorescence measurements in the Cy5 channel (for human ACTB) and the Texas-Red channel (for viral S-gene) were taken after 30 minutes of RT-LAMP.

FIG. 2 shows the evaluation of LAMP primers for non-S-gene mutations found in the Alpha variant.

FIG. 3 shows the evaluation of LAMP primers for S-gene mutations found in the Alpha variant.

FIG. 4 shows the screening of multiple sets of LAMP primers for the E484K mutation in the S-gene of SARS-COV-2.

FIG. 5 shows time courses of the fluorescence intensity in RT-LAMP assays containing different LANTERN probes: (a) Evaluation of a probe targeting the A1708D mutation in the ORF1ab gene of SARS-COV-2. 2E4 copies of in vitro transcribed RNA templates corresponding to A1708 or D1708 were used as template; (b) Evaluation of a probe targeting the E484K mutation in the S-gene of SARS-COV-2. 2E4 copies of in vitro transcribed RNA templates corresponding to E484 or K484 were used as template. 0.5U of Q5 High-Fidelity DNA Polymerase was also added in each reaction mix. Here, RT-LAMP reactions were performed without (−) or with (+) swarm primers; and (c) Assessing the effect of additional reverse transcriptase or Bst2 enzyme. In vitro transcribed S-gene templates (corresponding to E484 or K484) were spiked into 0.25 ng of PC9 RNA. 0.5 μM K484-targeting LANTERN probe was used with a S-gene LAMP primer set that excluded the swarm primers. 0.5U of Q5 High-Fidelity DNA Polymerase was also added in each reaction. Fluorescence in the Cy5 channel (for human ACTB) and the Texas-Red channel (for viral S-gene) was monitored over the course of the reactions.

FIG. 6 shows the conceptualization and development of the SNIPER probe: (a) Schematic of the SNIPER probe. Since the probe binds its target with perfect complementarity, the Bst polymerase may extend from it like a regular primer. To add the first nucleotide to the 3′ end of the probe, the Bst enzyme will cleave the fluorophore/quencher attached to the 3′ OH of the probe, thereby leading to a fluorescence signal. To better discriminate between matched and mismatched templates, the mismatch position is at the second nucleotide and a LNA residue is inserted close to the mismatched nucleotide, since LNA residues have a more rigid ribose sugar than canonical nucleotides and thus have a lower tolerance for mismatches; (b) Probes from 21-23nt long with LNA inserted at the −1 and/or −3 position were tested with 2E4 copies of wildtype (E484) or mutant (K484) template. No swarm primers were used. Fluorescence measurements here were taken after 30 minutes of RT-LAMP; (c) Probes from 21-23nt long with LNA inserted at the −1 and/or −3 position were tested with 2E4 copies of wildtype (E484) or mutant (K484) template. Swarm primers were added in the reactions. Fluorescence measurements here were taken after 30 minutes of RT-LAMP; (d) Analytical LoD of the SNIPER probe for synthetic RNA templates encoding the E484 or K484 version of the viral S-gene. Fluorescence measurements here were taken after 30 minutes of RT-LAMP; and (e) Analytical LoD of the SNIPER probe for lentiviruses packaged with either the E484 or K484 S-gene fragment. Lentiviruses were diluted in TE buffer with 0.1U/ul of Proteinase K and heated at 95° C. for 5 minutes prior to addition in the RT-LAMP reaction. Fluorescence measurements here were taken after 30 minutes of RT-LAMP.

FIG. 7 shows the optimization of displacement primers, which are known to be important for the kinetics of LAMP reactions. Three different B3 primers were evaluated using either (a) 20 copies or (b) 2 copies of synthetic S-gene (K484) RNA template per reaction.

FIG. 8 shows the evaluation of the effect of extra Bst2 enzyme on the performance of the method described herein. Here, 0.5 μM of 22nt LNAnt1 probe targeting the K484 template was used without swarm primers. 2E4 copies of in vitro transcribed RNA (either K484 or E484) were added in each sample as template. The RT-LAMP reactions were carried out without (−) or with (+) extra 8U Bst2 DNA polymerase.

FIG. 9 shows the incorporation of human internal control: (a) Effect of Q5 high-fidelity DNA polymerase on LNA-containing SNIPER probes. There was a slight increase in fluorescence signal for the mismatched template, but the Q5 enzyme appeared to encounter difficulty in cleaving the mismatched nucleotides when LNA residues were present; (b) Analytical LoD in 2-plex reactions with human ACTB internal control. Variable copies of synthetic viral RNA with K484 mutation and 0.25 ng of PC9 RNA were added in each reaction. Concentration of ACTB LAMP primers were reduced to 0.2λ with or without swarm primers. 0.5UM or 1 μM of ACTB SUN-conjugated LANTERN probe and 0.5U of Q5 DNA Polymerase were added for detection of the ACTB amplicon. Fluorescence in the Cy5 channel (S-gene) and the HEX/SUN channel (ACTB) were taken after 30 minutes of RT-LAMP; (c) Assessing the effect of extra reverse transcriptase instead of extra Bst2 on 2-plex RT-LAMP reactions. 1 μM of LANTERN ACTB probe was used without swarm primers; (d) Evaluating the effect of different Bst DNA polymerases on 2-plex RT-LAMP reactions. Bst3.0, Turbo Bst2.0, and IsoPol+ (8U each) were tested. Variable copies of synthetic RNA with K484 mutation and 0.25 ng of PC9 RNA were added in each reaction. 1 μM of LANTERN ACTB probe was used without swarm primers. 0.5U of Q5 DNA Polymerase was also added for detection of the ACTB amplicon. Fluorescence in the Cy5 channel (S-gene) and the HEX/SUN channel (ACTB) were taken after 30 minutes of RT-LAMP.

FIG. 10 shows the Assay sensitivity for saliva samples: (a) Workflow of the SNIPER diagnostic test using the probe described herein for saliva samples without RNA extraction. To lyse the virus, each sample is treated with proteinase K and heated at 95° C. for 5 minutes before being transferred into an RT-LAMP reaction mix containing sequence-specific probes against viral genes and a human internal control. The sample tube is then incubated at 65° C. for up to 30 minutes before fluorescence readings are taken. The fluorescence signals can also be monitored over the course of the reaction in a real-time PCR instrument; (b) and (c) Analytical LoD for saliva samples spiked with variable amounts of lentivirus packaged with either the K484 or E484 S-gene fragment. Each RT-LAMP reaction was supplemented with 8U of (b) Turbo Bst2 or (c) IsoPol+. Every reaction mix also contained 0.5 μM of SNIPER viral S-gene (K484) probe, 1 μM of LANTERN human ACTB probe, and 0.5U of Q5 DNA Polymerase. Fluorescence in the Cy5 channel (S-gene) and the HEX/SUN channel (ACTB) was taken after 30 minutes of RT-LAMP; (d) Analytical LoD for contrived saliva specimens treated with proteinase K and 50 mM EDTA. RT-LAMP reactions were supplemented with 8U of IsoPol+; (e) Analytical LoD for contrived saliva specimens processed using a commercially available ZeroPrep Saliva Collection Kit. RT-LAMP reactions were supplemented with 8U of IsoPol+; (f) Analytical LoD for contrived saliva specimens processed using the ZeroPrep kit. RT-LAMP reactions were supplemented with 8U of IsoPol+. Here, the probe used contained two LNA residues at the nt1 and nt3 positions, whereas the probes used in (b)-(e) contained only a single LNA residue at the nt3 position.

FIG. 11 shows results of a 3-plex assay for detection of SARS-COV-2 variants: (a) Testing a 21nt FAM-conjugated SNIPER probe targeting the wildtype (E484)S-gene of SARS-COV-2. Here, RT-LAMP reactions were supplemented with 8U of Bst2 enzyme; and (b) Evaluation of a 3-plex assay containing FAM-conjugated S-gene (E484) SNIPER probe, Cy5-conjugated S-gene (K484) SNIPER probe, and SUN-conjugated ACTB LANTERN probe. 2E4 copies of synthetic viral RNA was spiked into 0.25 ng of PC9 RNA to simulate purified RNA samples. Here, RT-LAMP reactions were supplemented with 8U of IsoPol+.

FIG. 12 shows the screening of multiple sets of LAMP primers for the K417N mutation in the S-gene of SARS-COV-2.

FIG. 13 shows SNIPER probes for different SARS-COV-2 mutations: (a) Evaluation of various SNIPER probes for detection of K417N in the S-gene; (b) Evaluation of various SNIPER probes for detection of A1708D in the ORF1ab gene; (c) Evaluation of A1708D probes in the presence of Q5 high-fidelity polymerase; (d) Evaluation of various SNIPER probes for detection of R203M in the N-gene (left panel), A119-120 in the ORF8 gene (middle panel), and D950N in the S-gene; (e) Evaluation of various SNIPER probes for detection of E484A (left panel) and Q498R (right panel) in the S-gene. For (a)-(e), 0.5 μM of probes were evaluated with 2E4 copies of the wildtype or mutant templates. Fluorescence measurements here were taken after 30 minutes of RT-LAMP; (f) Evaluation of swarm primers for human ACTB gene amplification. RT-LAMP reactions were supplemented with 8U of IsoPol+ and tested against 0.25 ng PC9 RNA. Every reaction contained 0.5 μM SNIPER probe targeting the LoopB region of the ACTB amplicon; and (g) Analytical LoD for mutant synthetic RNA added with 0.25 ng PC9 RNA. RT-LAMP reactions were supplemented with 8U of IsoPol+. Every reaction contained 0.5M ACTB probe and 0.5 μM of the corresponding SNIPER probe for each mutation. ACTB LAMP primers were added at 0.2λ concentration with only one swarm primer (F1c).

FIG. 14 shows the screening of multiple sets of LAMP primers for different Delta and Omicron mutations.

FIG. 15 shows the development of a 2-plex, 3-probe assay: (a) Schematic showing how two different viral probes function. The FAM-conjugated loop probe will fluoresce in the presence of any viral strain as it has been designed to target a highly conserved region, while the Cy5-conjugated SNP probe is only activated when a specific mutation is present. Note that it is also possible for the stem region to be chosen as the variant-independent site if the selected mutation is in the loop region instead; (b) Testing of loop probes designed to detect amplicons of the Beta (E484K), Alpha (A1708D), Delta (R203M), and Omicron (E484A) strains. 2E4 copies of either mutant or wildtype synthetic RNA were used as template; and (c) Analytical LoD for different 3-probe assays. Variable copies of viral template were spiked into 0.25 ng PC9 RNA.

FIG. 16 shows time courses for RT-LAMP reactions with or without a loop primer that may interfere with the corresponding SNIPER probe.

FIG. 17 shows the evaluation of 3-probe assays with unpurified samples: (a,c) K484 probe was tested on three different lentiviruses mimicking wildtype, Beta, or Omicron; and (b,d) A484 probe was tested on three different lentiviruses mimicking wildtype, Beta, or Omicron. Data shown in (a) and (b) were obtained using WarmStart LAMP reagents stored frozen at −20° C., while data shown in (c) and (d) were obtained using lyophilized LAMP reagents stored at room temperature.

FIG. 18 shows the general applicability of SNIPER probes: (a) Evaluation of SNIPER probes of different length for detection of the K417N mutation in the S-gene of SARS-COV2. 0.5UM of probes were evaluated with 2E4 copies of the wildtype or mutant templates. Fluorescence measurements here were taken after 30 minutes of RT-LAMP; and (b) Evaluation of SNIPER probes of different length for detection of the V600E mutation in the human BRAF gene. Fluorescence readings here were taken after 30 minutes of RT-LAMP.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention. Embodiments described below in context of the detection probe are analogously valid for the respective methods and kits, and vice versa. The various embodiments are not necessarily mutually exclusive, as some embodiments can be combined with one or more other embodiments to form new embodiments.

To address the limitations of existing approaches to nucleic acid molecule detection, there is provided an alternative sequence-specific detection method as described herein that provides a sensitive and specific assay for detection of a desired target and that can be readily programmed to identify genetic variants. The sequence-specific detection method described herein is based, in part, on the strong polymerization activity of DNA polymerases (e.g. Bst polymerase) and a specifically developed detection probe.

In particular, the method according to various embodiments described herein may be used for determining the presence and/or quantity of a target nucleic acid molecule containing a point mutation or SNP, in a broad range of samples using a loop-mediated isothermal amplification assay and a specifically developed detection probe.

As used herein, the “sample” may be any suitable sample selected from but not limited to environmental samples (e.g., soil samples, dirt samples, garbage samples, sewage samples, industrial effluent samples, air samples, water samples from a variety of water bodies such as lakes, rivers, ponds etc), food samples (e.g. samples from food intended for human or animal consumption such as processed foods, raw food material, produce, legumes, meats, fish, seafood, nuts, beverages, drinks, fermentation broths, and/or a selectively enriched food matrix comprising any of the above-listed foods, infant formulas, infant food, etc.), or biological samples. A biological sample may refer to a sample obtained from a subject that may be any eukaryotic or prokaryotic source and may be, for instance, in the form of a single cell, in the form of a tissue, or in the form of a fluid. In various embodiments, the biological sample may be biological fluids, including blood, plasma, serum, saliva and the like. In various embodiments, the biological sample may be derived from a subject, suffering from or suspected of suffering from a disease, for example an infectious disease, the subject preferably being a mammal, for example a human. In various embodiments, the biological sample may be derived from a cell culture media. In various embodiments, the subject may also be an animal or plant. In various embodiments, the subject may be a human. If the method is used for pathogen detection, any sample type useful and known for such purpose may be used, such as saliva.

In various embodiments, the sample may not be subjected to any nucleic acid purification or extraction step prior to use in the methods described herein.

In various embodiments, the sample may be subjected to heat-inactivation in order to obtain a crude extract of the target nucleic acid molecule prior to use in the methods described herein. In various embodiments, the sample may be heated at about 95° C. for about 5 minutes alone prior to use in the methods described herein. In various embodiments, the sample may be treated with proteinase K at room temperature for 1 minute and then heated at about 95° C. for about 5 minutes. For example, the heat and proteinase K treatment may help to release the target nucleic acid molecule from inside a viral particle contained within said sample. In various embodiments, the sample may be treated with proteinase K and/or heat treatment.

As used herein, the term “target” refers to the target nucleic acid to be detected but further encompasses the amplicons produced by the loop mediated isothermal amplification reaction that include sequences of the target that are recognized by the primers and/or the detection probes. Accordingly, when reference is made to a target that is bound by primers and/or detection probes, this term typically relates to the amplicons as produced in the loop mediated isothermal amplification reaction, as these are more prevalent than the original target nucleic acid. “Amplicons”, as used herein, relate to the amplified products generated starting from the template, i.e. the original target nucleic acid.

In various embodiments, the target nucleic acid molecule may be a nucleic acid sequence on a single strand of nucleic acid. In various embodiments, the target nucleic acid molecule may be a portion of a gene, a regulatory sequence, genomic DNA, cDNA, RNA including mRNA and rRNA, or others. In various embodiments, the target nucleic acid molecule may be DNA or RNA, whereby the detection probe described herein may be used to detect both DNA and RNA targets unlike RNase H-dependent methods.

In various embodiments, the target nucleic acid molecule may be genetic variants that contain at least one nucleotide point mutation, single nucleotide variations (SNVs), or single nucleotide polymorphisms, relative to a reference sequence, such as a wild-type nucleic acid molecule. In various embodiments, the methods according to various embodiments described herein may be utilised in determining the presence or quantity of a genetic variant that contains at least one known nucleotide point mutation (i.e. SNP) relative to a reference sequence. Accordingly, in various embodiments, the method described herein may be used for detecting and quantifying a point mutation in a target nucleic acid molecule in a sample using the detection probe described herein.

The term “mutation” refers to physical or structural change of a nucleotide sequence caused in a wild-type gene, and change of a transcript (RNA) or a translation product (protein) derived therefrom. The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “mutant,” or “polymorphic” refers to a gene or gene product that displays modifications in sequence when compared to the wild-type gene or gene product, whereby an allele having the mutation in an allele population of the same type of genes is designated as a “mutant gene”. Examples of the mutation include substitution, deletion and addition of one or plural nucleotide bases, including single nucleotide polymorphisms (SNP), point mutations or single nucleotide variations (SNVs). It is noted that naturally-occurring mutants may be isolated; these are identified by the fact that they have altered characteristics when compared to the wild-type gene or gene product. As used herein, the term “point mutation” refers to the identity of the nucleotide present at a site of a mutation in the mutant copy of a genomic locus. As used herein, a SNP is a type of point mutation that occurs at the same genomic locus between different individuals in a population and constitutes a substitution of a single nucleotide at a specific position, that is generally present in a sufficiently large fraction of the population (e.g. 1% or more). In various embodiment, the point mutation may be a SNP.

Accordingly, in various embodiments, the methods described herein may be used for determining the presence or quantity of a point mutation, such as a single nucleotide polymorphism (SNP), in a target nucleic acid molecule in a sample. That is, the target nucleic acid may comprise at least one point mutation or SNP known to be characteristic or indicative of a genetic variant. In this context, a “known” point mutation refers to a known mutation that can be present in a specific position of a target nucleic acid molecule. The specific position refers to a prescribed position in the target nucleic acid molecule. The point mutation may be any of substitution, deletion and addition of one or plural bases, preferably a substitution. In various embodiments, the point mutation may be a SNP, whereby the SNP may be defined as having been previously recorded in either RefSeq RNA or dbSNP at NCBI.

In various embodiments, the detection probe described herein may comprise a nucleotide sequence fully complementary to a nucleotide sequence of the probe binding site of the target nucleic acid comprising the known point mutation or SNP and comprises a nucleotide complementary to the known point mutation or SNP.

In various embodiments, the nucleotide sequence of the probe binding site comprises at least one point mutation. In various embodiments, the nucleotide sequence of the probe binding site comprises a single point mutation, preferably a nucleotide substitution. In this regard, the primers used in the method described herein may be designed to amplify the loci in the target nucleic acid molecule comprising the at least one point mutation relative to a reference wild-type nucleotide sequence.

In various embodiments, the at least one point mutation may be a single point mutation, preferably a SNP.

In various embodiments, the nucleotide base in the detection probe that is complementary to the known point mutation in the target nucleic acid molecule is positioned at the penultimate (second from last (nt2)) nucleotide base relative to the 3′-end of the detection probe.

It is envisioned that the methods according to various embodiments described herein may be utilised in determining the presence of a point mutation in a target nucleic acid molecule for application in one or more of the following areas:
Cancer diagnosis/screening for specific cancer mutations;
Viral variant detection/surveillance of infectious diseases;
Discrimination of pathogenic from non-pathogenic bacteria;
Disease risk stratification based on ClinVar/dbSNP database;
Genetic disease screening; and.
Pharmacogenomic testing.

In various embodiments, the methods described herein may be utilised in determining the presence of a point mutation indicative of a disease or condition in a subject, more particularly a biomarker for said disease or condition in said target nucleic acid. For example, the method described herein may be used to detect variants of the human BRAF gene indicative of multiple neoplasms, whereby the target nucleic acid molecule may comprise a nucleotide sequence including a known nucleotide point mutation which causes an amino acid substitution at position V600.

In various embodiments, the methods described herein may be utilised in determining the presence or quantity of viral variants derived from wild-type viruses. In particular, the method described herein can be readily used to detect point mutations, preferably single nucleotide polymorphisms (SNPs) characteristic of said viral variants. Existing methods lack the intrinsic property to resolve single nucleotide differences within the target sequence. This feature is useful not only for the identification of wild type viruses themselves but also for the detection of new viral variants that emerge over the course of a pandemic. In particular, the method according to various embodiments described herein may be used to formulate a rapid, sensitive, and highly specific diagnostic assay for the identification of viruses (such as COVID-19) and any variant derived from the wild-type viral strain.

In various embodiments, the target nucleic acid molecule may be a nucleic acid of a pathogen, optionally a human pathogen, preferably a bacterial, fungal, parasite or viral nucleic acid molecule or a cDNA reverse transcript of a bacterial, fungal, parasite or viral RNA. In various embodiments, the target nucleic acid molecule may be a nucleic acid of a coronavirus, influenza virus, paramyxovirus or enterovirus.

In various embodiments, the target nucleic acid molecule may be a nucleic acid of SARS-CoV-2 virus. For example, the method described herein may be used to detect variants of SARS-COV-2, whereby the target nucleic acid molecule may comprise of a nucleotide sequence of SARS-COV-2 including a known point nucleotide mutation or SNP which causes an amino acid substitution at a position selected from but not limited to D3, R203, N501, R521, A570, K417, E484, Q498, P681, D950, S982, T1001, or A1708, or causes an amino acid deletion selected from but not limited to A119-120 or 43675-3677. In particular, the substitution of N501Y can be found in the Alpha, Beta, Gamma, and Omicron variants; K417 is mutated to N in the Beta, Delta, and Omicron variants and to T in the Gamma variant; and E484 is mutated to K in the Beta and Gamma variants and to A in the Omicron variant31. Specifically, the substitutions T10011, A1708D, R521, D3L, A570D, P681H, S982A and deletion 43675-3677 can be found in the Alpha variant; the substitutions K417N, E484K can be found in the Beta variant; the substitutions R203M, D950N and deletion A119-120 can be found in the Delta variant; and the substitutions E484A and Q498R can be found in the omicron variant.

In various embodiments, the methods described herein may be readily adapted to detect any infectious agent or disease outbreak in the future and genetic variants derived therefrom, as well as being adaptable for other areas and uses that require detecting the presence, absence or quantity of a point mutation or SNP in a target nucleic acid in a sample.

In particular, point-of-care or point-of-need detection methods according to various embodiments described herein may enable sensitive, rapid, affordable, simple-to-use detection and assisted diagnosis of infectious diseases and is also readily-adaptable to detect and assist in diagnosing genetic variants of such infectious diseases. In various embodiments, the methods according to various embodiments described herein represents a sensitive and specific RT-LAMP assay that can be readily programmed to identify genetic variants.

For example, methods according to various embodiments described herein may be used for the detection and assisted diagnosis of infectious diseases, such as COVID-19 and variants including Alpha, Beta, Delta, and Omicron, and permit testing to be exponentially scaled up around the world. The COVID-19 pandemic has highlighted a vital need for sensitive, simple-to-use, affordable, and readily adaptable diagnostic assays for detecting SARS-COV-2 and any emerging variants. Commonly deployed quantitative real-time PCR assays and antigen rapid tests either require specialized facilities or lack an inherent ability to distinguish between variants respectively. In contrast, the methods according to various embodiments described herein have been based on a fluorescent probe-based RT-LAMP method that is highly sensitive to single nucleotide mismatches, allowing the specific identification of different SARS-COV-2 variants, including Alpha, Beta, Delta, and Omicron.

As evidenced by the working examples, a panel of specifically designed probes were evaluated on patient samples and shown to achieve near-perfect concordance with sequencing-based variant classification. Moreover, the methods according to various embodiments described herein are readily-adaptable and may be quickly reconfigured to detect different pathogens and their variants, providing a useful tool to address any future infectious disease outbreak.

In various embodiments, there is provided a detection probe that is designed specifically for use in the methods described herein. In various embodiments, the detection probe may be a single-stranded probe, preferably a single-stranded DNA probe, that recognises a probe binding site within the target nucleic acid and target amplicons. In particular, the detection probe may comprise a nucleic acid sequence complementary to the target nucleic acid and target amplicons comprising the at least one point mutation or SNP, more particularly a region of the target nucleic acid that is amplified such that the probe binding site is located in the amplicons formed by the LAMP reaction. In various embodiments, the detection probe may comprise a nucleotide complementary to the point mutation or SNP. In various embodiments, the detection probe may comprise a nucleotide complementary to a known single point mutation, preferably known SNP in the target nucleic acid and target amplicons.

In various embodiments, the detection probe described herein does not have any probe binding site constraints and so may be designed to bind with and placed at any available position on an amplicon. In various embodiments, the probe binding site may lie between the binding sites of primers used in the LAMP reaction. Accordingly, the detection probe described herein may be much more straightforward to design than molecular beacons and strand displacement probes that are used in other assays.

In various embodiments, said probe binding site may be different from and non-overlapping with any primer binding sites used in the LAMP setup. In particular, the detection probe described herein may be designed to be separate and distinct from the primers to rule out spurious by-products and does not interfere with the process of LAMP. Thus, the detection probe described herein may act as an extra specificity check to guard against spurious amplicons and serves as an add-on feature to any existing LAMP assay.

In various embodiments, the detection probe described herein may be a separate oligonucleotide and is not an extension of any primer used in the LAMP reaction, and therefore is much less likely to interfere with the amplification process. The detection probe described herein being independent from the primers used for amplification allows the user of the methods to utilize optimal primer binding sites without the need to be close to the location of a point mutation or SNP to be detected. In contrast, primer-specific methods would restrict the user to design the primers such that the 3′ end (or 5′ end for F1c) must be at the point mutation or SNP position. This may result in the user being forced to utilize sub-optimal LAMP primers.

In this regard, the detection probe described herein does not restrain the design of LAMP primers, and provides an independent specificity check, as well as allowing for multiplexed detection within one reaction tube.

In various embodiments, the detection probe may be designed such that it can hybridize to the probe binding site on the amplicons formed under loop mediated isothermal amplification assay conditions to form a double-stranded probe: target complex. The hybridization is typically achieved by designing the detection probe sequence such that the nucleotides contained therein can form Watson-Crick base pairs with the designated sequence of the probe binding site in the amplicons. Generally, when reference is made herein to “complementarity”, it is meant that the respective sequence can form Watson-Crick base pairs with its designated target or counterpart, wherein the term “fully complementary” as used herein refers to the respective sequence stretch being complementary over the entire length of the respective region, i.e. all of the bases in the nucleotide sequence of the detection probe need to form Watson-Crick base pairs with its counterpart sequence of the probe binding site, so long as the probe can hybridize to the probe binding site.

In various embodiments, the detection probe described herein may be designed to hybridize to said target amplicons under LAMP assay conditions and form a double-stranded probe: target complex. In this regard, suitable LAMP assay conditions are known in the art.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon.

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. For example, the melting temperature can be calculated using the “Analyze function”, whereby the melting temperature (Tm) is the temperature at which an oligonucleotide duplex is 50% in single-stranded form and 50% in double-stranded form. An Oligo Analyzer estimates Tm from the nearest-neighbor two-state model, which is applicable to short DNA duplexes,

$${T}_{M} ( ° C . ) = \frac{Δ H °}{Δ S ° + R ln [ oligo ]} - 273.15$$

where ΔH° (enthalpy) and ΔS° (entropy) are the melting parameters calculated from the sequence and the published nearest neighbor thermodynamic parameters, R is the ideal gas constant (1.987 calK-1mole-1), [oligo] is the molar concentration of an oligonucleotide, and the constant of −273.15 converts temperature from Kelvin to degrees of Celsius³².

The term “stringent conditions”, as used herein, is the “stringency” which occurs within a range from about Tₘ−5° C. (5° C. below the melting temperature (Tm) of the detection probe) to about 20° C. to 25° C. below Tₘ.

Accordingly, the detection probe may be fully complementary to the nucleotide sequence of the probe binding site with all base pairs matching perfectly and thus the detection probe does not comprise any mismatched base pairing with the nucleotide sequence of the probe binding site in the amplicons, such that the detection probe may hybridize to the target nucleic acid or target amplicons with perfect complementarity base pairing.

In various embodiments, the fully complementary double-stranded probe: target complex may have a higher melting temperature (Tm) in comparison to a non-fully complementary double-stranded probe: target complex in which the target nucleic acid comprises at least one mismatched nucleotide. In this regard, the mismatched nucleotide may correspond to the site of the point mutation (i.e. SNP) to be detected in the sample, that is the mismatched nucleotide may reflect the wild-type sequence or a different genetic variant that is not the “target” and is not desired to be detected. In various embodiments, the melting temperature (Tm) of the fully complementary double-stranded probe: target complex may be higher by about 10, 8, 7, 6, 5, 4, 3, 2, 1.5, 1, or 0.5° C. in comparison to the non-fully complementary double-stranded probe: target complex, this may be termed as the Tₘ differential (See Tables 2-4 below). In various embodiments, the melting temperature (Tm) of the fully complementary double-stranded probe: target complex may be greater than about 3° C. in comparison to the non-fully complementary double-stranded probe: target complex.

In contrast to the detection probe described herein, other probes have been previously developed to contain a deliberately mismatched nucleotide at the 3′ end (i.e. not fully complementary), so that a high-fidelity DNA polymerase can be used to digest away the unhybridized mismatched nucleotide, leading to unquenching of a fluorophore. One such detection probe was termed LANTERN probe³⁰. In principle, the LANTERN probe may be used for variant detection as the alternative allele with a SNP or mutation will create 3′ end mismatches that trigger the exonuclease activity of the polymerase, while the wildtype allele will form an intact double-stranded stem upon probe binding. However, through investigative study it was found that a perfectly matched complex could also give rise to an appreciable fluorescence signal, which was difficult to eliminate. Hence, the detection probe described herein was developed and based on a different mechanism (i.e. to that of the LANTERN probe) that reduced the off-target signal to near-background levels, thereby yielding a high mutant-to-wildtype output ratio. The detection probe described herein, may be termed as SNIPER (Single Nucleotide Polymorphism Effective Reporter), for application in LAMP assays that could specifically identify genetic variants at high sensitivity, such as with some of the major SARS-COV-2 variants.

In various embodiments, the detection probe may comprise at least one modified nucleotide base and is fully complementary to the nucleotide sequence of the probe binding site. The modified detection probe may have higher specificity (i.e. binding affinity) to the sequence of the probe binding site in the amplicons and hybridizes more strongly as compared to an unmodified detection probe. That is, the detection probe described herein may have an increased melting temperature (Tₘ value) as compared to an unmodified detection probe having a basic skeleton with the same base sequence and the same nucleic acid fragment length. The inclusion of the modification may allow the detection probe to hybridize to the target nucleic acid more strongly as compared to an unmodified detection probe. In various embodiments, the detection probe described herein may be modified to include at least one modified nucleotide base in order to increase the binding affinity and Tₘ value of the probe for the target sequence at the probe binding site compared to a probe of the same sequence without the modification, under the same conditions for detection, e.g., such as amplification conditions, or stringent hybridization conditions. In various embodiments, the modified base may provide a Tₘ differential of about 15, 12, 10, 8, 7, 6, 5, 4, 3, 2, 1.5, 1, or 0.5° C., compared to a probe of the same sequence without the modification.

In various embodiments, the modified detection probe may have higher melting temperature (Tm) and/or specificity (i.e. binding affinity) to a fully complementary sequence of the probe binding site in the amplicons and hybridizes more strongly as compared to a non-fully complementary sequence of the probe binding site that contains a mismatched nucleotide base. In this regard, the mismatched nucleotide may correspond to the site of the point mutation (i.e. SNP) to be detected in the target nucleic acid molecule in the sample, that is, the mismatched nucleotide may reflect the wild-type sequence or a different genetic variant that is not the desired target to be detected. In various embodiments, the melting temperature (Tm) of the modified detection probe that is fully complementary to the target probe binding site may be higher by about 10, 8, 7, 6, 5, 4, 3, 2, 1.5, 1, or 0.5° C. in comparison to the modified detection probe that is non-fully complementary to the target probe binding site.

A “modified base” or other similar terms such as “nucleotide analogue” refers to a non-naturally occurring nucleobase or nucleosidic base, which can pair with a natural base (e.g., adenine, guanine, cytosine, uracil, and/or thymine) and/or can pair with a non-naturally occurring nucleobase or nucleosidic base. A modified base as defined herein may refer to a nucleotide modified at the backbone, internucleotide linkage, sugar or base moiety. Modifications at the backbone or internucleotide linkage moiety include peptide nucleic acid (PNA) and substitution of the phosphate group by phosphorothioate. Modifications at the sugar moiety include locked nucleic acid (LNA) and substitution of the 2′—OH group. Modifications of the base moiety include alterations of A, T/U, G and C.

In various embodiments, the detection probe may comprise at least one 3′ end (terminal) modified nucleotide base, wherein the detection probe may hybridize to the target amplicons under isothermal amplification assay conditions and form a double-stranded probe: target complex. In various embodiments, the at least one modified nucleotide base may be positioned close to or at the 3′ end of the detection probe.

In various embodiments, the at least one modified nucleotide base may comprise at least one LNA or PNA residue, or combinations thereof, to increase the Tₘ value of the detection probe as compared to a detection probe without inclusion of a LNA or PNA, and therefore the hybridization efficiency may be improved. Functionally, PNA and LNA are similar and may be incorporated into the detection probe described herein for the same purpose.

In various embodiments, the at least one modified nucleotide base may comprise at least one LNA residue. A “locked nucleic acid” LNA is nucleic acid having two ring structures in which in a sugar-phosphoric acid skeleton, an oxygen atom in the 2-position and a carbon atom in the 4-position of ribose are bonded to each other by methylene crosslinking. The locked ribose conformation enhances base stacking and backbone pre-organization, which significantly increases the hybridization properties (e.g., increases thermodynamic stability and melting temperature) of oligonucleotides. Accordingly, LNA nucleotides may increase the sensitivity and specificity of detection probes. When oligonucleotide containing LNA anneals to DNA, the double-stranded conformation is changed and thereby the thermal stability is improved. Since LNA has high avidity to nucleic acid as compared to common oligonucleotide, more reliable and stronger hybridization can be achieved depending on, for example, the conditions for designing nucleotide. Such oligomers are synthesized chemically and are commercially available.

In various embodiments, the at least one modified nucleotide base may comprise at least one PNA residue. A “peptide nucleic acid” (PNA) has a structure in which a deoxyribose main chain of oligonucleotide has been substituted with a peptide main chain. Examples of the peptide main chain include a repeating unit of N-(2-aminoethyl) glycine bonded by an amide bond. Examples of the base to be bonded to the peptide main chain of PNA include, but not limited to, naturally-occurring bases such as thymine, cytosine, adenine, guanine, inosine, uracil, 5-methylcytosine, thiouracil, and 2,6-diaminopurine as well as artificial bases such as bromothymine, azaadenine, and azaguanine. PNA oligomers are known to exhibit higher binding specificity for complementary DNAs than regular DNA. In other words, a PNA-DNA base mismatch is much more destabilizing than a comparable mismatch in a DNA-DNA duplex.

In various embodiments, the at least one LNA or PNA residue may be positioned close to or at the 3′ end of the detection probe. The increase in the number of LNA or PNA residues around the 3′ end of the probe, may increase the stability of the probe. Moreover, LNA and PNA residues are resistant to nucleases and proteases, such that the inclusion of LNA and/or PNA within the detection probe described herein may lead to improved nuclease or protease resistance. Accordingly, in various embodiments, the inclusion of modified bases or nucleotide analogues within the detection probe described herein may improve nuclease and/or protease resistance. Further, the inclusion of phosphorothioate bonds may render probes more resistant to nuclease digestion.

In addition, the inclusion of LNA and/or PNA residues may allow the detection probes described herein to be compatible with the additional use of LANTERN probes (i.e. single-stranded DNA probes containing a 3′-end nucleotide mismatch) as a proofreading polymerase enzyme (i.e. high-fidelity DNA polymerase with 3′ exonuclease activity) to be added to the LAMP reaction mixture will have more difficulty in cleaving off the LNA/PNA residues and thus will not disrupt the functioning of the detection probe described herein.

In various embodiments, the at least one LNA or PNA residue may comprise a single 3′ end LNA or PNA residue. In various embodiments, the single 3′ end LNA or PNA residue may be positioned at the 5th (nt5), 4th (nt4), 3rd (nt3), 2nd (nt2), or 1st (nt1) nucleotide relative to the 3′ end of the detection probe, whereby the 1st (nt1) nucleotide represents the last 3′-end nucleotide of the detection probe sequence where a fluorophore or quencher may be attached thereto. “nt” in this context is an abbreviation of “nucleotide”. In this regard, these nucleotide positions may be represented as 5′-(ntN) (nt5) (nt4) (nt3) (nt2) (nt1)-3′, whereby the at least one LNA or PNA residue may be positioned at any one or more nucleotide base between and inclusive of the 1st to 5th base from the 3′-end of the probe. (ntN) denotes the remaining number of nucleotides of the probe sequence from the 5′-end, with N being any number between 5-45 (i.e. in a probe that is 10-50 nucleotides in length) such that N+5=the length of the probe.

In various embodiments, the single 3′ end LNA or PNA residue may be positioned at the first (nt1), second (nt2), or third (nt3) nucleotide relative to the 3′ end of the detection probe. In various embodiments, the single 3′ end LNA or PNA residue may be positioned at the third (nt3) nucleotide relative to the 3′ end of the detection probe. In various embodiments, the single 3′ end LNA or PNA residue may be positioned at the second (nt2) nucleotide relative to the 3′ end of the detection probe. In various embodiments, the single 3′ end LNA or PNA residue may be positioned at the first (nt1) nucleotide relative to the 3′ end of the detection probe.

in various embodiments, the at least one 3′ end LNA or PNA residue may comprise two 3′ end LNA or PNA residues. In various embodiments, the two LNA or PNA residues may be placed in consecutive positions and next to each other, or may be spaced apart from each other by 1 nucleotide base. In various embodiments, the two 3′ end LNA or PNA residues may be the first (nt1) and third (nt3) nucleotide relative to the 3′ end of the detection probe (nt1+3). In various embodiments, the two 3′ end LNA or PNA residues may be the second (nt2) and third (nt3) nucleotide relative to the 3′ end of the detection probe (nt2+3). In various embodiments, the two 3′ end LNA or PNA residues may be the third (nt3) and fourth (nt4) nucleotide relative to the 3′ end of the detection probe (nt3+4), in the event that the mutation is a deletion of more than 2 nucleotides.

Accordingly, in various embodiments, the at least one LNA or PNA residue may comprise a LNA or PNA residue at the first, second, third and/or fourth, preferably third, or second and third, or first and third, or third and fourth nucleotide position relative to the 3′ end of the detection probe.

In various embodiments, a LNA or PNA residue may be placed:
(a) adjacent or immediately next to (nt1 or nt3) the nucleotide in the detection probe that is complementary to the point mutation at the penultimate base (nt2);
(b) immediately either side (nt1 and nt3) of the nucleotide in the detection probe that is complementary to the point mutation at the penultimate base (nt2);
(c) at the nucleotide (nt2) in the detection probe that is complementary to the point mutation at the penultimate base (nt2);
(d) a combination of (a) and (c); or
(e) at two nucleotides upstream, towards the 5′ end, (nt3 and nt4) of the nucleotide in the detection probe that is complementary to the point mutation at the penultimate base (nt2), whereby the point mutation is a deletion of one or more nucleotides.

In various embodiments, in addition to the at least one LNA or PNA residue, the detection probe may further comprise at least one additional modified base. The additional modified nucleotide base may increase the detection probes melting temperature (Tm) or binding affinity, improve nuclease or protease resistance, eliminate probe secondary structure formation and/or reduce fluorophore quenching. In various embodiments, the at least one additional modified base may include but is not limited to 8-aza-7-deazaguanosine 2,6-Diaminopurine, 5-hydroxybutynl-2′-deoxyuridine, 5-Methyl deoxycytidine, and bases with a phosphorothioate bond incorporated. Hence, for probes with low on-target fluorescence, these modified bases may be used to increase their Tₘ and binding affinity in LAMP reactions. Also, they can be used in AT-rich probes to boost the Tₘ and binding affinity.

In various embodiments, the inclusion of the at least one additional modified bases or nucleotide analogues within the detection probe described herein may eliminate probe secondary structure formation and/or reduce fluorophore quenching. For example, the at least one modified nucleotide base may comprise 8-aza-7-deazaguanosine (in place of guanosine) to prevent the formation of non-canonical base pairs thereby eliminating secondary structure formation that impairs the synthesis of oligonucleotides containing G tracts.

Accordingly, in various embodiments, the inclusion of LNA/PNA residues and additional modified bases or nucleotide analogues may perform one or more of the following functions in relation to the detection probe:
increase the binding affinity;
increase the Tₘ value;
improve nuclease resistance;
improve protease resistance;
eliminate probe secondary structure formation; and
reduce fluorophore quenching.

In various embodiments, the detection probe may range in length from 10 nucleotides to 50 nucleotides, preferably 12 to 30 nucleotides. In various embodiments, the detection probe is 14-30 nucleotide bases in length. In various embodiments, the detection probe is 14-25 nucleotide bases in length. In various embodiments, the detection probe is 14-23 nucleotide bases in length, preferably 14-21 nucleotide bases in length. In various embodiments, the detection probe is 14, 15, 16, 17, 18, 19, 20, 21, 22, 23, 24 or 25 nucleotide bases in length.

In various embodiments, the detection probe may be conjugated or attached to any fluorophore or quencher, more particularly any fluorophore-quencher pair. This is unlike the LUX primer and HyBeacon probe, where only a subset of fluorophores with self-quenching properties may be used. Through the use of different fluorophore-quencher pairs, the methods described herein may be used to simultaneously detect several different targets in a single reaction tube in a multiplexing method.

The term “Fluorophore” as used herein, refers to a moiety that emits fluorescence when excited by light of the proper wavelength, whereas “quenchers” refer to a moiety that suppresses the fluorescence emission of the fluorophore. In a fluorescence resonance energy transfer (FRET) pair the two members of the pair influence each other as long as they are in close spatial proximity, for example when bound to the same molecule, with this influence becoming less pronounced the farther apart the two members are. This allows to detect the difference between an intact probe, where both moieties are in close proximity, and a cleaved probe, where each fragment comprises one member of the pair so that they are no longer in close proximity to each other. In a typically fluorophore-quencher pair, the quencher suppresses fluorescence of the fluorophore if both are present in the same molecule. Once both get separated by cleavage of the molecule such that both are no longer present in the same molecule, the influence of the quencher is reduced so that the fluorescence of the fluorophore is detectably increased.

In various embodiments, the detection probe may be conjugated or attached to a quencher-fluorophore pair at opposite ends of the probe and at a distance that allow the quencher to quench the fluorophore signal. The quencher-fluorophore pair may be positioned such that they may interact in the intact non-cleaved probe and selected such that the fluorescence signal changes upon cleavage of the probe. In the detection probes described herein, this is typically given, even if both are positioned on opposing 5′ and 3′ ends, respectively, of the probe, or vice versa.

In various embodiments, either the fluorophore or the quencher is attached to the 3′ end of the probe, that is, the fluorophore or the quencher may be conjugated to the last nucleotide at the 3′ end of the probe. In various embodiments, the quencher may be attached to the 5′ end of the detection probe and the fluorophore may be attached to the 3′ end of the detection. In various embodiments, the fluorophore may be attached to the 5′ end of the detection probe and the quencher may be attached to the 3′ end of the detection.

As the fluorophore or quencher may be conjugated to the 3′ end of the probe, it will be released from the probe after cleavage, thereby producing a fluorescent signal. In principle, the position of the fluorophore and quencher may also be swapped, in which case the quencher separates from the probe after cleavage. In particular, the fluorophore or quencher conjugated to the 3′ OH of the probe will be cleaved off by the DNA polymerase (i.e. Bst polymerase) after hybridization of the detection probe to the target amplicon, thereby separating the fluorophore and quencher from one another, which then results in a fluorescence signal to be measured and optionally quantified by well-known means in the art.

Accordingly, in various embodiments, there is provided a method for determining the presence, or quantity, of a point mutation, preferably single nucleotide polymorphism (SNP), in a target nucleic acid molecule in a sample using loop mediated isothermal amplification (LAMP), the method comprising:
(a) combining a LAMP reaction mixture, DNA polymerase with 5′→3′ polymerase activity, and a detection probe with the sample (suspected of containing the target nucleic acid molecule),
   wherein the LAMP reaction mixture comprises a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule,
   wherein the detection probe recognises a probe binding site within target amplicons,
   wherein the detection probe is a single-stranded probe comprising:
      a nucleotide sequence fully complementary to a nucleotide sequence of the probe binding site comprising the point mutation;
      a nucleotide complementary to the point mutation at the penultimate base relative to the 3′ end of the detection probe; and
      a locked nucleic acid (LNA) or peptide nucleic acid (PNA) residue at the first, second, third and/or fourth, preferably third, or second and third, or first and third, or third and fourth nucleotide position relative to the 3′ end of the detection probe,
   wherein the detection probe comprises a quencher-fluorophore pair at opposite ends of the probe at a distance that allow the quencher to quench the fluorophore signal,
   wherein the detection probe can hybridize to said target amplicons under LAMP assay conditions and form a double-stranded probe: target complex,
(b) amplifying the target nucleic acid molecule by LAMP under suitable assay conditions that allow:
   i. generation of the target amplicons;
   ii. hybridization of the detection probe to the target amplicons to form the probe: target complex; and
   iii. cleavage of the detection probe by the DNA polymerase to release the quencher or fluorophore; and
(c) detecting and optionally quantifying the released quencher or fluorophore to determine the presence or quantity, of the point mutation in the target nucleic acid molecule in the sample.

In various embodiments of the loop-mediated isothermal amplification (LAMP) method described herein, the “target nucleic acid”, refers to the target nucleic acid to be detected but further encompasses the amplicons and concatemers produced by the LAMP reaction that include sequences of the target that are recognized by the inner primers, the loop primer(s) and the detection probe. Accordingly, when reference is made to a target that is bound by the LAMP primers or the detection probes, this term typically relates to the amplicons and concatemers as produced in the LAMP reaction, as these are more prevalent than the original target nucleic acid. “Amplicons” or “concatemers”, as used interchangeable herein, relate to the amplified products generated starting from the template, i.e. the original target nucleic acid, and dumbbell starting structure produced from the inner primers in a first part of the LAMP reaction. These structures contain multiple repeats of the relevant sequence elements described above.

As used herein, the terms “LAMP” or “loop-mediated isothermal amplification”, refer to a method that is performed at an essentially constant temperature without the need for a thermocycler. In LAMP, the target sequence is typically amplified at 60 to 65° C. using either two or three sets of primers (i.e. 4 to 6 primers). Typically, 4 different primers are used to identify 6 distinct regions on the target gene, which adds highly to the specificity. An additional “loop primer” or pair of “loop primers” can further accelerate the reaction. Due to the specific nature of the action of these primers, the amount of DNA produced in LAMP is considerably higher than PCR based amplification. The LAMP method is described in U.S. Pat. No. 6,410,278 B1 and U.S. Pat. No. 7,374,913 B2. Generally, the method uses two inner primers (forward inner primer=FIP and backward inner primer=BIP), two outer primers (F3 and B3) which recognize six distinct regions in the target, and optionally one or two, preferably two, loop primers (loop forward=LF and/or loop backward=LB) to boost amplification efficiency. If two loop primers are used, one is preferably a loop forward primer and the other a loop backward primer. The inner primers comprise a target complementary region (typically referred to as F2 and B2) that facilitates hybridization and 5′ thereto a sequence that is identical to a sequence in the target nucleic acid located upstream (5′) relative to the sequence of the target bound by the target complementary region of the inner primer (typically referred to as F1c and B1c). Elongation of the inner primer by the polymerase thus creates a sequence comprising regions of self-complementarity in that the target-identical sequence on the 5′ end of the inner primer (B1c) can, after elongation, bind to the synthesized sequence downstream of the target-complementary region of the inner primer (referred to as B1) and act as a primer for further extension. The outer primers bind to a target region in the target nucleic acid that lies downstream (i.e. 3′) to the target region bound by the inner primers (referred to as F3c and B3c) and thus are responsible for the displacement of the elongated inner primer sequences from the template strand. The elongated inner primers are recognized and hybridized by the other primer of the inner primer pair and thus the dumbbell structured starting amplicons are generated. The dumbbell shape includes the “loop” regions as the rounded portions with “stem” regions as the central handlebar, whereby the loop and stem regions are single-stranded, thereby allowing the probe to hybridize. The “loop” refers to the sequence between the 5′ B1c/F1c region on the primer and the complementary sequence on the target nucleic acid B1, and the “stem” region refers to the sequence of the target nucleic acid between F1c and B1c complementary sequences on the target nucleic acid. The point mutations or SNPs are preferably positioned in the loop and/or stem regions as they are single-stranded in the dumbbell structure, hence, allowing probe binding. The dumbbell structures are used for the following amplification, with the amplicons taking the form of concatemers. The principle of LAMP amplification and reagents to use are common general knowledge for those skilled in the art.

In various embodiments of the method described herein, and in accordance with the established principles of LAMP, the LAMP reaction mixture may comprise a LAMP primer set of 4 to 6 primers, comprising two inner primers (FIP and BIP), two outer primers (F3 and B3), and optionally one or two loop primers (LF and/or LB). While it is known that the loop primer(s) increase(s) amplification efficiency, these are optional and not essential for carrying out the LAMP method. It is however preferred that one or two, preferably two, loop primers are included in the methods of the invention.

In various embodiments, the LAMP primers may be designed to generate amplicons containing one or more pre-selected (pre-determined) and known point mutation relative to a reference sequence (e.g. wild-type sequence). In various embodiments, the LAMP primers may be designed such that the selected and known point mutation are not present in the primer binding sites. This allows both wildtype and genetic variant sequences to be amplified, and then subsequently the two may then be distinguished by the detection probe described herein.

The two inner primers used in the methods may thus each comprise a target complementary region on their 3′ end (F2 and B2) and a target identical region on their 5′ end (F1c and B1c), where in the target nucleic acid the sequence (i.e. primer binding site) recognized by the target complementary region of the inner primers (termed F2c or B2c) lies 3′ to the sequence identical to the target identical sequence on the 5′ end of the inner primers (said sequence in the target termed F1c and B1c).

Accordingly, the two outer primers each comprise a target complementary region (F3 and B3), wherein in the target nucleic acid the sequence (i.e. primer binding site) targeted by the target complementary region of the outer primers (termed F3c and B3c) are located 3′ to the sequence of the target nucleic acid targeted by the target complementary region of the inner primers. This allows displacement of the elongated inner primers necessary for generation of the dumbbell shaped starting structures needed for concatemer formation in the later stages of LAMP.

The one or two optional loop primers each comprise a target complementary region that recognizes a sequence (i.e. primer binding site) between the target complementary region on the 3′ end of the inner primers or the complement thereof (i.e. the F2 or B2 region) and the sequence complementary to the target identical sequence on the 5′ end of the inner primers or the complement thereof (i.e. the F1 or B1 region). The forward loop primers preferably bind between F1 and F2. Similarly, preferred binding for the backward loop primers is thus between B1 and B2. It may be preferred that the loop primer set comprises loop primers that bind between the F1 and F2 and loop primers that bind between the B1 and B2 regions of the amplicons.

in various embodiments, two additional primer sets may be utilized in addition to the two inner primers (FIP and BIP) and two outer primers (F3 and B3), and two loop primers (LF and LB). These two additional primer sets may include stem primers, which target the single-stranded region in the center of the dumbbell structure, and the swarm primers, which hybridize to the template strand opposite to that of FIP or BIP so as to reveal the binding sites for the inner primers.

In various embodiments, the primer set may further comprise two swarm primers including a forward swarm primer and a backward swarm primer. In various embodiments, the primer set may further comprise two stem primers including a forward stem primer and a backward stem primer. In various embodiments, the primer set may further comprise two swarm primers and two stem primers.

In various embodiments, the respective binding sites recognized by the LAMP primers and the detection probe are non-overlapping. Accordingly, the probe binding site for the detection probe may be different from the primer binding site of the LAMP primers, more preferably non-overlapping with the LAMP primer binding sites.

The LAMP method is characterized by generating unique stem-loop structures, which contain single-stranded regions. These single-stranded regions may provide ideal positions for single strand probe hybridization without the need to separate the double-stranded DNA either through heating or strand displacement enzymes. LAMP is performed isothermally and probe hybridization has been optimized to be carried out at the same temperature. Both of these enable the LAMP reaction and probe hybridization to occur simultaneously, thus greatly facilitating real-time probe-mediated detection and improving the detection speed. Therefore, in the methods described herein hybridization probes may target sequences in the single-stranded loop regions.

Accordingly, in various embodiments, the probe binding site may be in a loop region of the target amplicons formed by the LAMP and may be different from and non-overlapping with any one of the primer binding sites. Binding of the detection probe in the loop region may ensure that probe: target hybridization does not interfere with the ongoing amplification reaction mediated by the inner primers. Alternatively, in various embodiments, the probe binding site may be in a stem region of the target amplicons formed by the LAMP and may be different from and non-overlapping with any one of the primer binding sites.

In various embodiments, the target nucleic acid used as a template for the LAMP reaction may be any nucleic acid molecule. In various embodiments, the target nucleic acid molecule may be a nucleic acid of a pathogen, optionally a human pathogen, preferably a bacterial, fungal, parasite or viral nucleic acid molecule. In various embodiments, the target may be the viral RNA of SARS-COV-2, more particularly an S-gene fragment of SARS-COV-2 RNA.

In various embodiments of the methods for SARS-COV-2 detection, where the target is the S-gene of SARS-COV-2 and amino acid substitution E484K, the detection probe may comprise the nucleic acid sequence set forth in any one of SEQ ID Nos. 121-129 or a variant thereof having at least 90% sequence identity over the entire length. In various embodiments, the detection probe may comprise the nucleic acid sequence set forth in any one of SEQ ID Nos. 121-129 with a fluorophore conjugated to the 5′ end and a quencher conjugated to the 3′ end.

In various embodiments of the methods for SARS-COV-2 detection, where the target is the S-gene of SARS-COV-2 and amino acid substitution K417N, the detection probe may comprise the nucleic acid sequence set forth in any one of SEQ ID Nos. 130-132 or a variant thereof having at least 90% sequence identity over the entire length. In various embodiments, the detection probe may comprise the nucleic acid sequence set forth in any one of SEQ ID Nos. 130-132 with a fluorophore conjugated to the 5′ end and a quencher conjugated to the 3′ end.

When reference is made to sequence identity, this means that in a given nucleic acid molecule the respective nucleotide at a given position is identical to the nucleotide in a reference nucleic acid molecule at the corresponding position. The level of sequence identity is given in % and can be determined by an alignment of the query sequence with the template sequence.

The determination of the identity of nucleotide sequences is achieved by a sequence comparison. This comparison or alignment is based on the BLAST algorithm well-established and known in the art and is in principle carried out by aligning stretches of nucleotides in the nucleotide sequences with each other. Another algorithm available in the art is the FASTA algorithm. Sequence comparisons (alignments), in particular multiple sequence comparisons, can be generated using computer programs. Commonly used are for example the Clustal series or programs based thereon or the respective algorithms. Further possible are sequence comparisons (alignments) with the computer program Vector NTI® Suite 10.3 with the pre-set standard parameters, the AlignX-module of which is based on ClustalW. If not explicitly defined otherwise, sequence identity is determined using the BLAST algorithm.

Such a comparison allows determining the identity of two sequences and is typically expressed in % identity, i.e. the portion of identical nucleotides in the same or corresponding positions. If not explicitly stated otherwise, the sequence identities defined herein relate to the percentage over the entire length of the respective sequence, i.e. typically the reference sequence. If the reference sequence is 20 nucleotides in length, a sequence identity of 90% means that 18 nucleotides in a query sequence are identical while 2 may differ.

In various embodiments, the DNA polymerase may be any DNA polymerase which possesses 5′→3′ polymerase activity. In various embodiments, the DNA polymerase may be Bst polymerase or Bsm polymerase. In various embodiments, the Bst polymerase may be selected from Bst3 polymerase, Bst2 polymerase and IsoPol+. In particular, the Bst polymerase is itself used for signal generation, which comes with several benefits, whereby no secondary enzyme that may potentially affect LAMP amplification is required for fluorescent signal generation as the probe (i.e. fluorophore or quencher) is cleaved by the polymerase.

In various embodiments, no additional enzyme other than the DNA polymerase is needed to be added to step (a), whereby any additional enzyme may interfere with the LAMP reaction or itself be affected by the reaction conditions.

In various embodiments, step (a) may further comprise one or more additional LAMP reagents that are readily known to those skilled in the art and in accordance with the principles of the LAMP method. In particular, step (a) may further comprise reverse transcriptase, buffer, water, salts, nucleotides, divalent cations (e.g., Mg⁺⁺), or enhancing agents (e.g., betaine, dimethyl sulfoxide, ethylene glycol, glycerol, formamide, 7-deaza-2′-deoxyguanosine 5′-triphosphate, 2′-deoxyinosine 5′-triphosphate, or 1,2-propanediol).

In various embodiments, step (a) may further comprise pyrophosphatase. In various embodiments, the pyrophosphatase may be in an amount in the range of 0.20 to 1U, preferably about 0.5U (or 0.02U/μL). In various embodiments, the pyrophosphatase may be a thermostable inorganic pyrophosphatase (TIPP). In various embodiments, the pyrophosphatase may be Inorganic pyrophosphatase (PPase).

In various embodiments, the combined mixture in step (a) may be incubated for any suitable time (e.g., about 20 to about 60 minutes) at a suitable temperature (e.g., from about 60° C. to about 65° C.) to promote amplification under step (b) consistent with the LAMP method and protocol known in the art. In particular, the mixture in step (a) may be incubated under suitable assay conditions known in the art and appreciated by the skilled person to allow for the generation of the target amplicons; hybridization of the detection probe to the target amplicons to form the probe: target complex; and cleavage of the detection probe by the DNA polymerase to release the quencher or fluorophore.

In various embodiments, in step (c) detection and optional quantification of the fluorescence signal readout from the released quencher or fluorophore (if the target is present in the sample) may be carried out using well-known techniques in the art. In particular, the released quencher or fluorophore may be detected and quantified by any suitable means known in the art.

In various embodiments, the background noise in the method described herein is low in the absence of the intended target nucleic acid being in the sample. Accordingly, the method described herein may exhibit very low background if the template is not a perfect match (i.e. not fully complementary), with even a single nucleotide difference being sufficient to reduce the signal to near background levels.

Moreover, if the detection probe described herein does not find its intended target, the fluorophore and quencher will remain intact on the same molecule and there will be minimal fluorescence signal regardless of oligonucleotide structure. In contrast, probes such as molecular beacons used in existing methods are designed to be non-fluorescent only when they are in a double-stranded conformation. As there is a delicate balance between hairpin stability and target hybridization, existing methods relying on such probes often encounter delayed fluorescence signals when the hairpin is too stable or high background noise when the hairpin is prone to melting, especially in LAMP where the operating temperature of 65° C. is relatively high.

Besides fluorescence readouts, in various embodiments, a lateral flow readout may also be used to detect cleavage of detection probes. In such embodiments, the detection probe may be labelled on both ends with markers that are recognized by antibodies. Examples of such markers include, without limitation, antigens including fluorescent markers that simultaneously function as antigen. Concrete examples include, without limitation, biotin, FITC and digoxigenin. In the lateral flow detection, the sample may be typically run on a capillary bed after being put on a first element of the lateral flow strip, the so-called sample pad. It then migrates to the second element, typically the conjugate pad is typically stored the so-called detection conjugates, for example in a dried format together with a matrix that allows the binding reaction between the target molecule (e.g., an antigen) and its chemical partner (e.g., antibody) that has been immobilized. As the sample fluid dissolves the conjugates and the matrix, the sample and conjugate mix while flowing through the porous structure. In this way, the analyte binds to the detection conjugates while migrating further through the capillary bed. This material has one or more areas (often called stripes) where a third or further “capture” molecule has been immobilized. By the time the sample-conjugate mix reaches these stripes, analyte has been bound by the detection conjugates and the “capture” molecule binds the complex. After a while, when more and more fluid has passed the stripes, complexes accumulate and the stripe-area changes color. Typically, there are at least two stripes: one (the control) that captures any detection conjugate and thereby shows that reaction conditions and technology worked fine and one that contains a specific capture molecule and only captures those conjugates which are complexed with an analyte molecule. After passing these reaction zones, the fluid enters the final porous material, the wick, that simply acts as a waste container.

In various embodiments, each lateral flow strip may comprise gold-conjugated IgG antibodies against the fluorophore near the sample pad, antibodies against the quencher immobilized at the control line, and antibodies against IgG immobilized at the test line. In the case of a target-free sample, the probe will stay intact such that when the reaction is loaded on the strip, the gold-conjugated IgG first binds to the fluorophore and then the entire IgG-probe complex is captured at the control line. Consequently, a dark band is observed only at the control line. However, in the case of a sample containing the target, the polymerase will cleave off the fluorophore such that when the reaction is loaded on the strip, the gold-conjugated IgG still binds to the fluorophore, but now, some of the gold will not be deposited at the control line as the fluorophore is free. Instead, the IgG-fluorophore complex continues flowing along the strip to the test line, where it is captured by the anti-lgG antibodies. Consequently, a dark band is observed at the test line.

The method described herein is thus compatible with both a fluorescent readout and a lateral flow readout. In contrast, existing detection methods are only capable of providing a fluorescent readout. Accordingly, in various embodiments, the detection method in step (c) may be lateral flow detection and/or fluorescence detection.

In various embodiments, the method described herein is also straightforward to utilize for multiplex detection of several different targets. In particular, the method described herein can be readily deployed for simultaneous detection of multiple distinct targets simply by the use of two or more detection probes described herein with different fluorophore-quencher combinations. This is unlike existing methods such as PEI-LAMP, where it is difficult to interpret a mixture of colours within the precipitate. For example, the method described herein can detect both SARS-COV-2 and a human internal control in the same reaction tube by using two different fluorophores.

Accordingly, the method described herein may be adapted into a multiplexing method and used for determining the presence or optionally quantity of a point mutation in two or more target nucleic acid molecules in the sample, wherein the method uses one or more primer sets and one or more detection probes designed for each target nucleic acid molecule or for multiple target nucleic acid molecules.

In various embodiments, the method and multiplexing method described herein may additionally include an internal control to target one or more control nucleic acid molecules.

In various embodiments, the method described herein may additionally include an internal control LAMP primer set and control detection probe configured to target one or more control nucleic acid molecules, as well as an additional DNA polymerase other than the Bst polymerase. In particular, to verify that the sample has been correctly processed for use in these methods and to reduce false negative results, it may be advantageous to additional employ the use of an internal control. Thus, in various embodiments, the methods described herein may further comprise amplifying a control nucleic acid molecule in the sample and detecting the amplified control nucleic acid molecule. The “control nucleic acid molecule” is different to the target nucleic acid molecule and may be any nucleic acid that is expected to be present in all of the samples tested. For example, when a sample is from a human patient, a human gene product can be used as a control nucleic acid, such as the human beta actin (ACTB) gene. Following amplification, the control nucleic acid molecule can be detected using a control detection probe.

Accordingly, in various embodiments, step (a) in the method described herein may further comprise a second DNA polymerase, a control LAMP primer set and a control detection probe to detect the presence of a control nucleic acid molecule in the sample for use as an internal control; step (b) may further comprise amplifying the control nucleic acid molecule in the sample and step (c) may further comprise detecting the amplified control nucleic acid molecule.

In various embodiments, the control detection probe may be a LANTERN probe and the second DNA polymerase has 3′-5′ exonuclease activity such as a high-fidelity polymerase. In this regard, the DNA polymerase having 3′-5′ exonuclease activity does not interfere with or disrupt the function of the SNIPER detection probe described herein since it is unable to cleave the LNA or PNA residues in the absence of a mismatched base pairing and the Bst polymerase lacks this 3′-exonuclease activity. Accordingly, the method described herein may additionally include LANTERN probes and accompanying reaction reagents in the same LAMP reaction mixture as the SNIPER detection probe. In various embodiments, the control nucleic acid molecule may be human ACTB.

In various embodiments, there is provided the use of the detection probe as described herein for determining the presence of point mutations, preferably single nucleotide polymorphisms (SNPs), in a target nucleic acid molecule in a sample using loop mediated isothermal amplification (LAMP). All embodiments disclosed above in relation to the detection probe and the method described herein similarly apply to this use.

In another aspect, there is provided a kit for determining the presence, or quantity, of a point mutation, preferably a single nucleotide polymorphism (SNP) in a target nucleic acid molecule in a sample using loop mediated isothermal amplification (LAMP), the kit comprising: a LAMP reaction mixture comprising a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule; a DNA polymerase with 5′→3′ polymerase activity; and a detection probe that recognises a probe binding site within target amplicons, wherein the detection probe is a single-stranded probe comprising: a nucleotide sequence fully complementary to a nucleotide sequence of the probe binding site of the target nucleic acid comprising the SNP and comprising a nucleotide complementary to the SNP at the penultimate base; and a locked nucleic acid (LNA) or peptide nucleic acid (PNA) residue at the first, second, third and/or fourth, preferably third, or second and third, or first and third, or third and fourth nucleotide position relative to the 3′ end of the detection probe, wherein the detection probe comprises a quencher-fluorophore pair at opposite ends of the probe at a distance that allows the quencher to quench the fluorophore signal, wherein the detection probe can hybridize to said target amplicons under LAMP assay conditions and form a double-stranded probe: target complex.

In various embodiments, the kit may comprise internal controls and associated reagents that allow for the detection of a control nucleic acid molecule, such as an internal control LAMP primer set and control detection probe configured to target the control nucleic acid molecule, as well as an additional DNA polymerase other than the DNA polymerase with 5′→3′ polymerase activity.

All embodiments disclosed above in relation to the detection probe and the method described herein similarly apply to the kit.

### EXAMPLES

### Materials and Methods

### Synthesis of synthetic viral RNA

For SARS-COV-2, the S-gene, ORF8 and N-gene fragment was amplified by PCR from Addgene plasmid #153895 using Q5 High-Fidelity DNA Polymerase (New England Biolabs). The ORF1ab fragment was ordered as a gBlock from Integrated DNA Technologies (IDT) and cloned into a TOPO vector. To facilitate in vitro transcription (IVT), the forward primers were appended at the front with the T7 promoter sequence (5′-TAATACGACTCACTATAGG-3′). Amplified products were gel extracted with the PureNA Biospin Gel Extraction Kit (Research Instruments). At least 50 ng of T7-containing PCR product was used as template for IVT with the HiScribe T7 Quick High Yield RNA Synthesis Kit (New England Biolabs). The reaction was incubated overnight at 37° C. for maximum yield. Following Dnase I digestion for 1 hour at 37° C., the RNA was purified with the RNA Clean & Concentrator −5 Kit (ZYMO Research), analysed by 2% TAE-agarose gel electrophoresis to assess RNA integrity, quantified with NanoDrop 2000, and stored at −20° C.

RT-LAMP reaction with SNIPER probes

All reactions were set up in a dedicated clean biosafety cabinet that was UV-irradiated before every use. Synthetic SARS-COV-2 RNA templates were serially diluted and amplified using the WarmStart LAMP Kit (New England Biolabs). 10× LAMP primer mix was prepared with concentration of 2 μM for F3, 2 μM for B3, 4UM for LF, and 4 mM for LB, 16UM for FIP, 16 μM for BIP, and 16UM for swarm F1c and swarm B1c. For the E484K/E484A target site, 4 μM for B3, 8 μM for LF, and 8 μM for LB is used. The RT-LAMP reaction was set up with 12.5 μl WarmStart LAMP Mastermix, 2.5 μl 10× LAMP primer mix, 2.5 μl 0.4M guanidine HCl, 0.25 μl thermostable inorganic pyrophosphatase (New England Biolabs), 1 μL of 8U/μL IsoPol® BST⁺ (ArcticZymes Technologies), 0.25 μl 50 μM SNIPER probe, 5 μl synthetic RNA, and RNase-free water such that the total reaction volume was 25 μl. For reactions with an internal control incorporated, 0.5 μl 10× ACTB primers (2 μM for F3 and B3, 16 μM for FIP and BIP, and 8UM for LF and LB) and 0.25 μl of 50 μM SNIPER probe for human ACTB were also added to the reaction mix. Alternatively, if LANTERN probes for ACTB were used, 0.25 μL of 2U/μL Q5 Polymerase were added with 0.25 μL of 100 μM ACTB LANTERN probe.

Subsequently, each sample tube was incubated for 40 minutes at 65° C. using a CFX96 Real-Time PCR Detection System (Bio-Rad) with fluorescence in the FAM, Cy5 and/or HEX channel measured every minute.

Table 1 below shows a list a primer sets that may be used in amplifying target nucleic acid molecules for use in LAMP assays with the detection probes described herein.

TABLE 1
Lists primer sets used

| | | | SEQ ID |
|---|---|---|---|
| Name | Primer | Sequence | NO: |
| S T7161 | F3 | CAGAAGTCCCTGTTGCTA | 1 |
| | B3 | CGAGGAGAATTAGTCTGAGTC | 2 |
| | FIP | TGCACGTGTTTGAAAAACATTAGAATTCATGCAGATCAAC | 3 |
| | | TTACTCC | |
| | BIP | GGCTGTTTAATAGGGGCTGAACATGATAACTAGCGCATAT | 4 |
| | | ACCTG | |
| | LF | CCTGTAGAATAAACACGCCAAGTA | 5 |
| | LB | CTCATATGAGTGTGACATACCC | 6 |
| S S982A | F3 | CCAATTTAATAGTGCTATTGGCA | 7 |
| | B3 | GCACTTCAGCCTCAACTT | 8 |
| | FIP | GCATTTTGGTTGACCACATCTTGAAAATTCAAGACTCACT | 9 |
| | | TTCTTCC | |
| | BIP | GCTTTAAACACGCTTGTTAAACAACTGTCAAGACGTGAAA | 10 |
| | | GGAT | |
| | LF | TTTTCCAAGTGCACTTGCT | 11 |
| | LB | AGCTCCAATTTTGGTGCAAT | 12 |
| S P681H | F3 | TTGGTGCAGGTATATGCG | 13 |
| set1 | | | |
| | B3 | ACATTGTACAATCTACTGATGTC | 14 |
| | FIP | AGGCAATGATGGATTGACTAGCTACTTATCAGACTCAGAC | 15 |
| | | TAATTCTCC | |
| | BIP | TAACTCTATTGCCATACCCACAAATTTGGTCATAGACACT | 16 |
| | | GGTAG | |
| | LB | TAGTGTTACCACAGAAATTC | 17 |
| | BIP2 | CTAATAACTCTATTGCCATACCCTTGGTCATAGACACTGG | 18 |
| | | TAG | |
| S P681- | F3 | ATTGGTGCAGGTATATGC | 19 |
| 701-716 | | | |
| Set 2 | | | |
| | B3 | TGGTAGAATTTCTGTGGTAAC | 20 |
| | FIP | TAGGCAATGATGGATTGACTAGCTCAGACTCAGACTAATT | 21 |
| | | CTCC | |
| | FIP2 | TGTAGGCAATGATGGATTGACTATCAGACTCAGACTAATT | 22 |
| | | CTC | |
| | BIP | CACTATGTCACTTGGTGCAGCTAATAGTAAAATTTGTGGG | 23 |
| | | TATGG | |
| | LF | TACACTACGTGCCCGCCGA | 24 |
| S P681- | F3 | AGGCTGTTTAATAGGGGC | 25 |
| 701-716 | | | |
| set3 | | | |
| | B3 | ATTTGTGGGTATGGCAATA | 26 |
| | FIP | TGAGTCTGATAACTAGCGCATATATCAACAACTCATATGA | 27 |
| | | GTGT | |
| | BIP | GGCACGTAGTGTAGCTAGTCAAGCAACTGAATTTTCTGC | 28 |
| | LF | CCTGCACCAATGGGTATGTC | 29 |
| | LB | TGCCTACACTATGTCACTTGG | 30 |
| S E484- | F3 | ACAATCTTGATTCTAAGGTTGG | 31 |
| N501set1 | | | |
| | B3 | ACTACTACTCTGTATGGTTGG | 32 |
| | FIP | CGGCCTGATAGATTTCAGTTGAAATTTACCTGTATAGATT | 33 |
| | | GTTTAGGAAG | |
| | BIP | GTAGCACACCTTGTAATGGTGTTGCAACACCATTAGTGGG | 34 |
| | | TTG | |
| | LF | CTCTCTCAAAAGGTTTGAGATTAG | 35 |
| | LB | GTTACTTTCCTTTACAATCATATGG | 36 |
| | F1c | CGGCCTGATAGATTTCAGTTGAAAT | 37 |
| | swarm | | |
| S E484- | F3 | CTCAAACCTTTTGAGAGAGA | 38 |
| N501set2 | | | |
| | B3 | GGTGCATGTAGAAGTTCA | 39 |
| | B3v2 | TCCACAAACAGTTGCTGGTGC | 40 |
| | B3v3 | CCACAAACAGTTGCTGGTGCATG | 41 |
| | FIP | GTAACAATTAAAACCTTCAACACCATATTTCAACTGAAAT | 42 |
| | | CTATCAGG | |
| | FIPv2 | GGAAAGTAACAATTAAAACCTATATTTCAACTGAAATCTA | 43 |
| | | TCAGG | |
| | BIP | TTCCTTTACAATCATATGGTTTCCAAAAGAAAGTACTACT | 44 |
| | | ACTCTGT | |
| | BIPv2 | TTACAATCATATGGTTTCCAACCCCAAAAGAAAGTACTAC | 45 |
| | | TACTCTGT | |
| | LF | CATTACAAGGTGTGCTACCG | 46 |
| | LB | CCCACTAATGGTGTTGGTTAC | 47 |
| | LB2 | AATGGTGTTGGTTACCAACC | 48 |
| | B1c | TTCCTTTACAATCATATGGTTTC | 49 |
| | swarm | | |
| S A570D | F3 | ACTGAGTCTAACAAAAAGTTTCT | 50 |
| | B3 | CCTGATAAAGAACAGCAACC | 51 |
| | FIP | CTGTGGATCACGGACAGCATCGCCTTTCCAACAATTTGGC | 52 |
| | BIP | ACTTGAGATTCTTGACATTACACCATGGTTAGAAGTATTT | 53 |
| | | GTTCCTGG | |
| | LF | AGTAGTGTCAGCAATGTCTCT | 54 |
| | LB | TGTTCTTTTGGTGGTGTCAG | 55 |
| S ΔHV | F3 | TTCTTTCACACGTGGTGT | 56 |
| | B3 | AGTGGAAGCAAAATAAACACC | 57 |
| | FIP | AGGTAAGAACAAGTCCTGAGTTGATTATTACCCTGACAAA | 58 |
| | | GTTTTCAG | |
| | BIP | TCTTTTCCAATGTTACTTGGTTCCAGACAGGGTTATCAAA | 59 |
| | | CCTCT | |
| | LB | GCTATACATGTCTCTGGGACCAATG | 60 |
| O8 R521 | F3 | TTCTTAGGAATCATCACAACTG | 61 |
| set2 | | | |
| | B3 | ATATCGATGTACTGAATGGGT | 62 |
| | FIP | GACACGGGTCATCAACTACATATGCACCAAGAATGTAGTT | 63 |
| | | TACAGTC | |
| | BIP | GAGTAGGAGCTAGAAAATCAGCACGATTTAGAACCAGCCT | 64 |
| | | CATCC | |
| O8 R521 | F3 | ACGCCTAAACGAACATGAA | 65 |
| set1 | | | |
| | B3 | AGAACCAGCCTCATCCAC | 66 |
| | FIP | GGTTGATGTTGAGTACATGACTGTACTTGTTTTCTTAGGA | 67 |
| | | ATCATCACA | |
| | BIP | ATATGTAGTTGATGACCCGTGTCCTAAAGGTGCTGATTTT | 68 |
| | | CTAGCT | |
| | LF | AACTACATTCTTGGTGAAATGCAGC | 69 |
| O1 T10011 | F3 | AACTCAATATGAGTATGGTACTG | 70 |
| | B3 | CTGAACAACTGGTGTAAGTTC | 71 |
| | FIP | TTGCTCTTCTTCAGGTTGAAGAGCTTACCAAGGTAAACCT | 72 |
| | | TTGGA | |
| | BIP | TTGGTCAACAAGACGGCAGTCATCTCTAATTGAGGTTGAA | 73 |
| | | CC | |
| | LF | AGCAGAAGTGGCACCAAAT | 74 |
| | LB | GAGGACAATCAGACAACTACTATTC | 75 |
| O1 A1708D | F3 | TGTTATCTTGCCACTGCA | 76 |
| | B3 | AGAATCTAAATTGGCATGTTGAA | 77 |
| | FIP | CCTTGCTCTGTAATAAGCATCTTGTTTAACACTCCAACAA | 78 |
| | | ATAGAGTT | |
| | FIPv2 | TGCTCTGTAATAAGCATCTTGTAGATTAACACTCCAACAA | 79 |
| | | ATAGAGTT | |
| | BIP | CTGGTGAAGCTGCTAACTTTTGTTGTTTCTCTAACATCAC | 80 |
| | | CTAAC | |
| | BIPv2 | GTGCACTTATCTTAGCCTACTGTTGTTTCTCTAACATCAC | 81 |
| | | CTAAC | |
| | BIPv3 | ACTTTTGTGCACTTATCTTAGCCTTGTTTCTCTAACATCA | 82 |
| | | CCTAAC | |
| | LF | AGAGCAGGTGGATTAAACTTC | 83 |
| | LB | GCCTACTGTAATAAGACAGTAGGT | 84 |
| O1 ASGF | F3 | GCTTTTGCAATGATGTTTGTC | 85 |
| 3675-3677 | | | |
| | B3 | GTTCTTGCTGTCATAAGGATT | 86 |
| | FIP | CCAACTAGCAGGCATATAGACCATACATTTCTCTGTTTGT | 87 |
| | | TTTTGTTACC | |
| | BIP | GTGATGCGTATTATGACATGGTTGGATGCATACATAACAC | 88 |
| | | AGTCTT | |
| | LF | AAGCTACAGTGGCAAGAGA | 89 |
| | LB | TGGTTGATACTAGTTTGTCTGG | 90 |
| N D3L | F3 | CGGTAATTATACAGTTTCCTGT | 91 |
| set2 | | | |
| | B3 | TGCATTTCGCTGATTTTGG | 92 |
| | FIP | GAACGAACAACGCACTACAAGATACCTTTTACAATTAATT | 93 |
| | | GCCAGG | |
| | BIP | GAGTATCATGACGTTCGTGTTGTGTCCATTATCAGACATT | 94 |
| | | TTAGTTTG | |
| N D3L | F3 | GTTGTTCGTTCTATGAAGACTT | 95 |
| set1 | | | |
| | B3 | CCGACGTTGTTTTGATCG | 96 |
| | FIP | TGGGGTCCATTATCAGACATTTTAGTAGAGTATCATGACG | 97 |
| | | TTCGTG | |
| | BIP | CGAAATGCACCCCGCATTACGCGTTCTCCATTCTGGTTAC | 98 |
| | LB | GACCCTCAGATTCAACTGGCA | 99 |
| S-K417 | F3 | TGTTATGGAGTGTCTCCTACT | 100 |
| set1 | | | |
| | B3 | TGAGATTAGACTTCCTAAACAATC | 101 |
| | FIP | GAGCGATTTGTCTGACTTCATCATGCTTTACTAATGTCTA | 102 |
| | | TGCAGAT | |
| | BIP | TACCAGATGATTTTACAGGCTGCCCACCAACCTTAGAATC | 103 |
| | | AAGA | |
| | LB | GTTATAGCTTGGAATTCTAACAATC | 104 |
| S-K417 | F3 | TGTCCTATATAATTCCGCATC | 105 |
| set2 | | | |
| | B3 | GAATCAAGATTGTTAGAATTCCA | 106 |
| | FIP | TGCATAGACATTAGTAAAGCAGAGCCACTTTTAAGTGTTA | 107 |
| | | TGGAG | |
| | BIP | TGATGAAGTCAGACAAATCGCTGCCTGTAAAATCATCTGG | 108 |
| | | TA | |
| | BIPv2 | GAGGTGATGAAGTCAGACAAATGCCTGTAAAATCATCTGG | 109 |
| | | TA | |
| | LF | ATCATTTAATTTAGTAGGAGAC | 110 |
| | LB | CAGGGCAAACTGGAA | 111 |
| | Swarm | TGCATAGACATTAGTAAAGCAGAG | 112 |
| | F1c | | |
| S-K417 | F3 | ATCTCTGCTTTACTAATGTCT | 113 |
| set3 | | | |
| | B3 | GAGATTAGACTTCCTAAACAATC | 114 |
| | FIP | ATCTTTCCAGTTTGCCCTGGGCAGATTCATTTGTAATTAG | 115 |
| | | AGG | |
| | BIP | TACCAGATGATTTTACAGGCTGCATTATAATTACCACCAA | 116 |
| | | CCTTAG | |
| | BIPv2 | GCTCCAGGGCAAACTGGAAAGCAGATTCATTTGTAATTAG | 117 |
| | | AGG | |
| | LF | GCGATTTGTCTGACTTCATCA | 118 |
| | LB | GCTTGGAATTCTAACAATCTTG | 119 |
| | Swarm | TACCAGATGATTTTACAGGCTGC | 120 |
| | B1c | | |

S = spike protein;
ΔHV = deletion of histidine and valine;
O1 = open reading frame 1;
O8 = open reading frame 8;
N = nucleocapsid protein;
ASGF = deletion of serine, glycine and phenylalanine.

### Preparation of pseudovirus

S-gene fragments were cloned into 3^{rd} generation lentiviral transfer plasmid using the NEBuilder HiFi DNA Assembly Master Mix (NEB #E2621). 24 hours prior to transfection, 1 X10⁶ cells are seeded into a 10 cm dish in fresh DMEM media. 10 μg of the transfer plasmid is then transfected along with 7.5 μg of pMDLg/pRRE (Addgene #12251), 2.5 μg of pRSV-Rev (Addgene #12253) and 2.5 μg of envelop plasmid pMD2.G (Addgene #12259) using the jetPRIMER Transfection Reagent (Polyplus Transfection®) according to manufacturer protocols. 24 hours post-transfection, the media is collected and replaced with PBS-free fresh DMEM media. At 48 hours post-transfection, the media is collected, combined with media collected earlier and filter sterilized through a 0.45 μm Acrodisc® syringe filter (Pall Corporation). The filtered media is then concentrated using Amicon® Ultra-15 Centrifugal Filter (Merck) and spun at 2900g for 25 minutes at 4° C. The concentrate is then transferred to a 1.5 mL tube and stored in −20° C.

Evaluation of SNIPER assay with contrived saliva samples

Lentivirus carrying SARS-COV2 S-gene fragment was serially diluted into healthy donor saliva. 8.3 μl of sample at each dilution was added with 1 μl Proteinase K (New England Biolabs) and 1 μL of 50 mM EDTA and vortexed for 1 minute at room temperature. Alternatively, 500 μl healthy donor saliva was added to 500 μl ZeroPrep Lysis Buffer (Veredus) before SARS-COV-2 virions at different dilutions were spiked in. The samples were then heated at 95° C. for 5 minutes before 4 μl was used for RT-LAMP.

### Results and Discussion

### Example 1: Evaluation of existing approaches

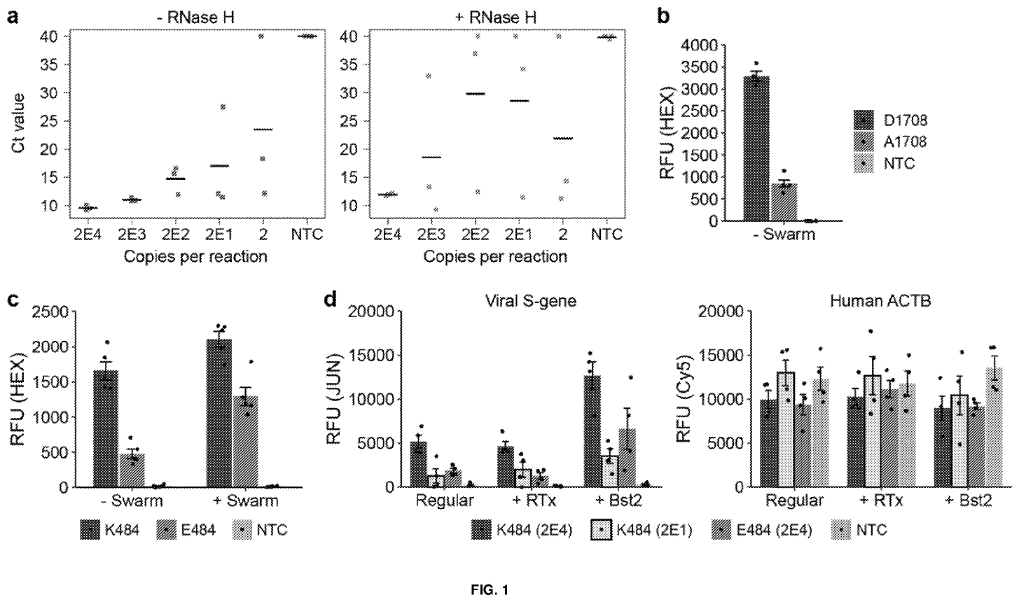

First, the addition of RNase H was investigated to see if it could improve the sensitivity of RT-LAMP. The enzyme has previously been reported to enhance the sensitivity of target detection in diagnostic tests that use recombinase polymerase amplification (RPA), likely by enhancing efficiency of the reaction through degradation of the RNA strand in the RT product, a RNA: DNA duplex19. However, it was discovered that the addition of RNase H reduced the sensitivity of RT-LAMP instead (FIG. 1a), possibly because the nuclease may cut the SARS-CoV-2 RNA template upon binding of the DNA primers. The result also suggests that RNase H-dependent methods20,21,28 cannot be used in RT-LAMP assays to detect genetic variants when the target material is RNA.

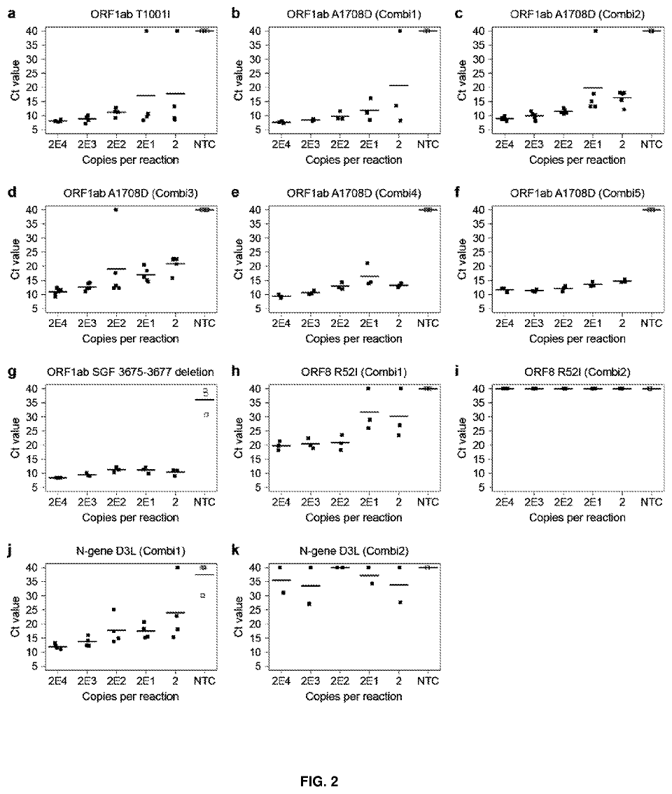

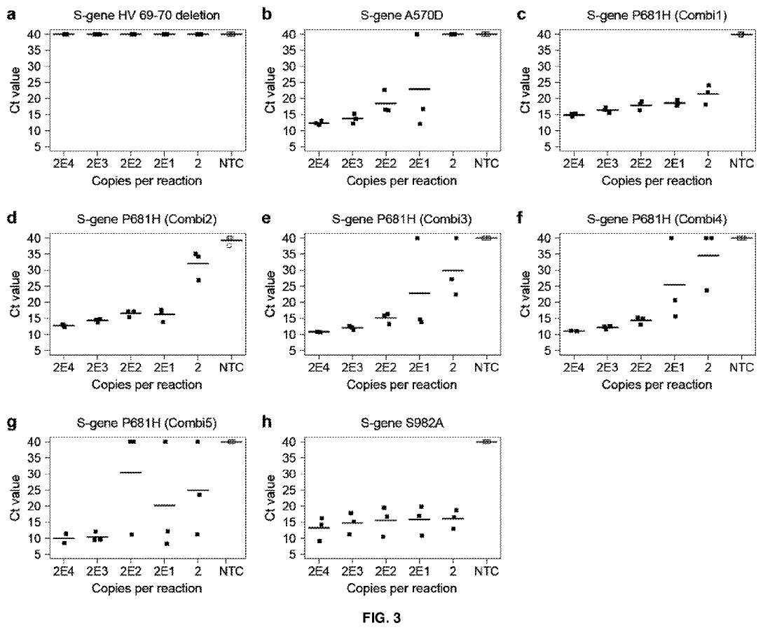

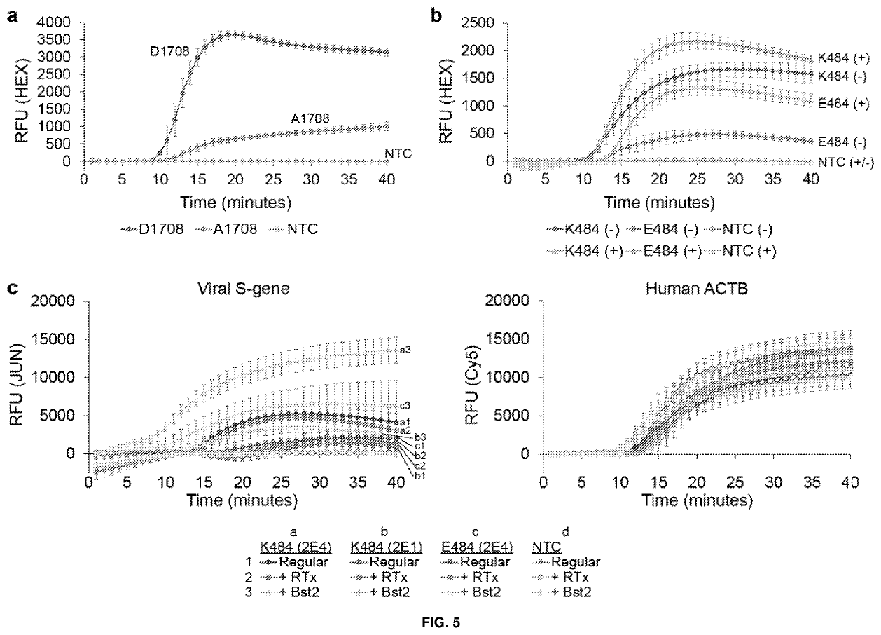

Next, the detection of specific SARS-COV-2 mutations was investigated using the methods and probes described herein. To this end, LAMP primer sets were screened for and designed using PrimerExplorer, that could robustly amplify loci containing known mutations in some of the major viral variants (FIG. 2-4). Two mutation sites were then selected for probe development. A LANTERN probe targeting the A1708D mutation in the ORF1ab gene gave a 3.9-fold higher signal for the variant than for the wildtype template (FIG. 1b and FIG. 5a). Similarly, another probe targeting the E484K mutation in the S-gene yielded a 3.6-fold higher signal for the variant than for the original template (FIG. 1c and FIG. 5b). Subsequently, the mutant-to-wildtype signal ratio was sought to be improved. In a bid to increase the fluorescence output for the intended target, several strategies were tried to boost the efficiency of RT-LAMP. However, the addition of swarm primers enhanced the fluorescence signal from both the mutant and wildtype templates (FIG. 1c and FIG. 5b). Furthermore, while increasing the amount of reverse transcriptase did not affect the fluorescence readings appreciably, increasing the amount of Bst2 polymerase also enhanced the signal from both the intended and the unintended targets (FIG. 1d and FIG. 5c). Hence, improvement of the output signal from the mutant template was not achieved without a concomitant increase in signal from the wildtype template.

### Example 2: Development of SNIPER probe

Although they did not trigger the proofreading activity of high-fidelity DNA polymerases, perfectly matched wildtype substrates still yielded a noticeable fluorescence signal in preliminary experiments with LANTERN probes. It was hypothesized that this unexpected signal arose because the Bst polymerase could extend from a perfectly bound probe by hydrolyzing and removing the quencher attached at the 3′ end of the probe before adding the first nucleotide. However, if there was a 3′ end mismatch between probe and target, the Bst enzyme itself should not be able to perform the polymerization reaction as it lacked a 3′-exonuclease activity.

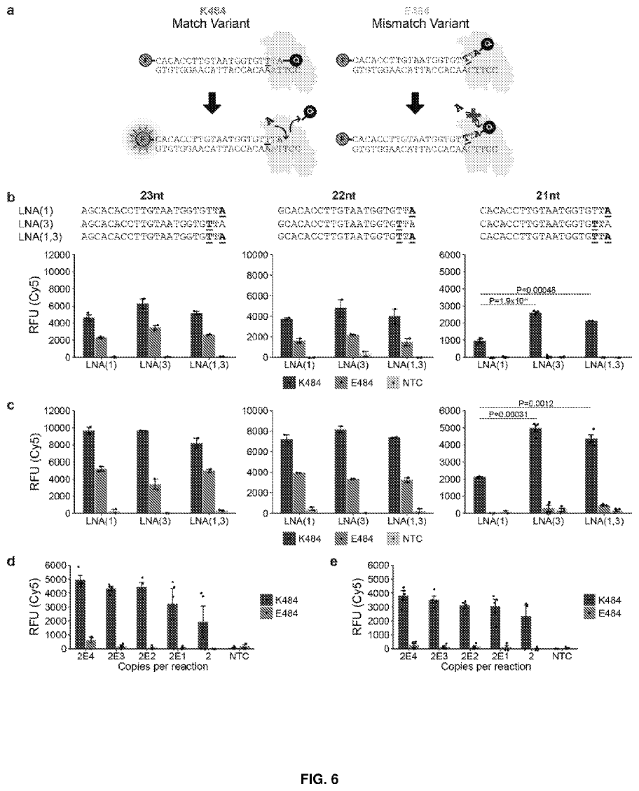

To test the hypothesis, new probes were designed targeting the E484K mutation in the S-gene, with Cy5 conjugated at the 5′ end and a quencher conjugated at the 3′ end. Unlike the LANTERN method, these probes were designed to match perfectly with the intended K484 target, whereas in the presence of the unintended E484 template, the probes would bind with a mismatch at the second last position from the 3′ end (FIG. 6). A mismatch at the penultimate position would also prevent the very last nucleotide at the 3′ end from hybridizing as well. Furthermore, as locked nucleic acid (LNA) monomers have a lower mismatch tolerance and higher affinity towards complementary bases than canonical nucleotides, LNA residues were placed at the first or/and third position from the 3′ end of our probes. As such, up to three nucleotides may potentially be prevented from hybridizing due to a single nucleotide mismatch. Table 2 below shows the melting temperatures of S-gene K484 SNIPER probes at different lengths with LNA nucleotides (bold underlined) inserted at first and/or third position from the 3′ end. Tₘ is calculated with OligoAnalyzer from IDT. Tₘ-PM denotes melting temperature towards perfect matched targets while Tₘ-MM denotes melting temperature towards mismatched targets.

TABLE 2

| | Sequence (underlined | SEQ ID | | Tm- | Tm- | |
|---|---|---|---|---|---|---|
| Name | bases denotes LNA) | NO | Length | PM | MM | ΔTm |
| 23-LNAnt1 | AGCACACCTTGTAATGGTTTAA | 121 | 23 | 65.3 | 65.8 | −0.5 |
| 23-LNAnt3 | AGCACACCTTGTAATGGTGTTAA | 122 | 23 | 67.9 | 65.4 | 2.5 |
| 23-LNAnt13 | AGCACACCTTGTAATGGTGTTAA | 123 | 23 | 67.3 | 65.4 | 1.9 |
| 22-LNAnt1 | GCACACCTTGTAATGGTGTTAA | 124 | 22 | 63.8 | 64.3 | −0.5 |
| 22-LNAnt3 | GCACACCTTGTAATGGTGTTAA | 125 | 22 | 66.5 | 63.7 | 2.8 |
| 22-LNAnt13 | GCACACCTTGTAATGGTGTTAA | 126 | 22 | 65.9 | 63.7 | 2.2 |
| 21-LNAnt1 | CACACCTTGTAATGGTGTTAA | 127 | 21 | 61.3 | 61.4 | −0.1 |
| 21-LNAnt3 | CACACCTTGTAATGGTGTTAA | 128 | 21 | 64 | 60.7 | 3.3 |
| 21-LNAnt13 | CACACCTTGTAATGGTGTTAA | 129 | 21 | 63.3 | 60.7 | 2.6 |

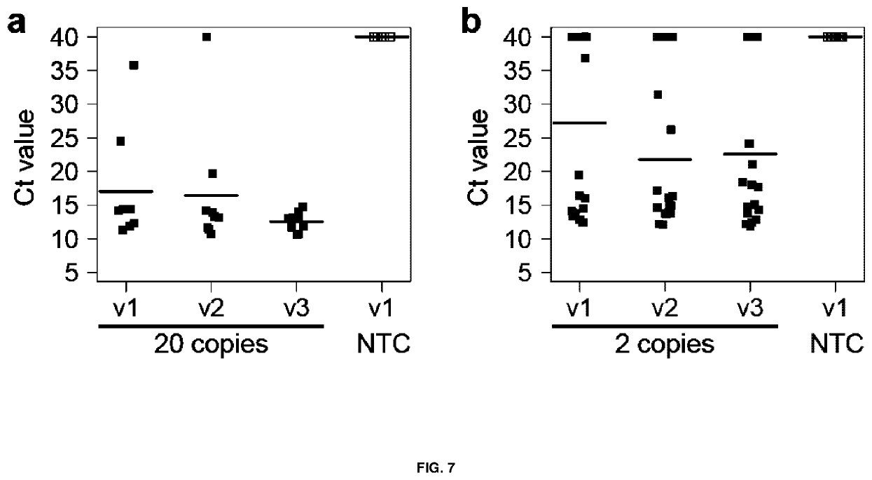

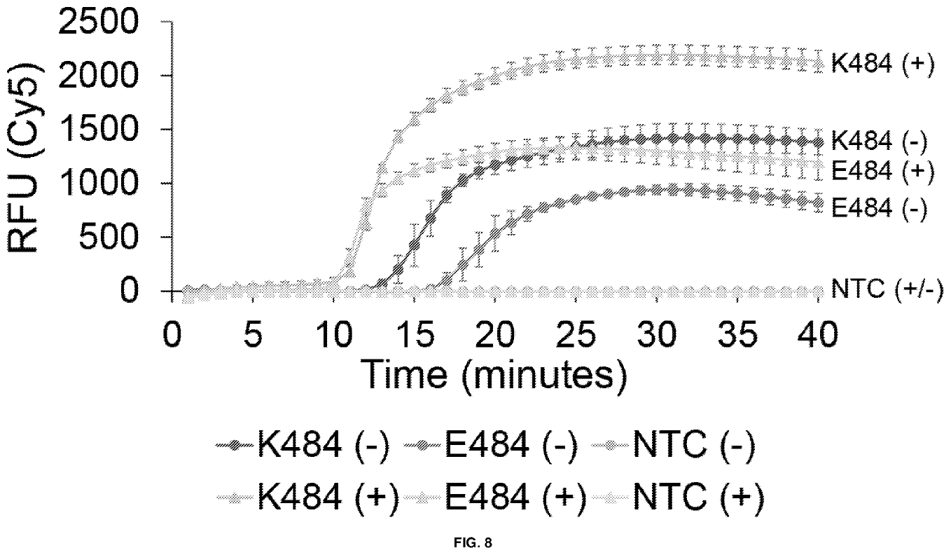

After optimizing the displacement primers (FIG. 7), the new probes were evaluated in RT-LAMP reactions with synthetic SARS-COV-2 RNA encoding either the E484 or the K484 sequence. No high-fidelity DNA polymerase was added, but each reaction was supplemented with additional Bst2 enzyme to boost fluorescence signals (FIG. 8). When the probe was 23-nucleotide (nt) long, the fluorescence readings of the intended K484 target were approximately double that of the mismatched E484 template (FIG. 6b). However, the undesired fluorescence generated from wildtype RNA was clearly too high. To reduce the signal of the mismatched E484 substrate, the probe length was shortened one nucleotide at a time, which lowered the melting temperature of the oligonucleotides. At a length of 21nt, the probes encountered sufficient difficulty in hybridizing with the mismatched template such that fluorescence from wildtype RNA was strongly attenuated to near-background levels. In contrast, the signal of the perfectly matched K484 target remained quite strong, with probes containing an internal LNA residue at the −3 position giving significantly higher fluorescence output than the probe containing only one LNA residue at the −1 position (P<0.005, one-sided Student's t-test). Notably, the mutant-to-wildtype signal ratio was at least 10 for all 21nt probes.

Next, the same set of probes was evaluated in RT-LAMP reactions containing swarm primers. Overall, it was found that addition of swarm primers enhanced the output signal of our probes at all lengths as expected (FIG. 6c). Excitingly however, at a probe length of 21nt, the fluorescence generated from the mismatched E484 template remained low even though the signal of the intended K484 target was greatly boosted. Again, in the presence of a perfectly matched substrate, probes with LNA at the −3 position emitted significantly higher fluorescence signals than the probe with a modified nucleotide at only the −1 position (P<0.005, one-sided Student's t-test). Hence, probes with LNA at the −3 position were further investigated and LNA-containing probes described herein were termed SNIPER (Single Nucleotide Polymorphism Effective Reporter).

To assess the sensitivity and accuracy of the detection probe described herein, RT-LAMP was performed with variable copies of mutant or wildtype viral template. Both in vitro transcribed SARS-COV-2 RNA (FIG. 6d) and packaged lentivirus expressing the relevant S-gene fragment (FIG. 6e) was performed. Overall, the SNIPER probe gave strong fluorescence signals for the intended K484 target but much weaker output signals for the mismatched E484 template, even at high copy numbers. Overall, the limit of detection (LoD) was close to 20 copies per reaction for both synthetic RNA and lentivirus. Collectively, the results show that regardless of viral titer, the SNIPER probe can discriminate between two substrates that differ by only a single nucleotide.

### Example 3: Incorporation of a human internal control

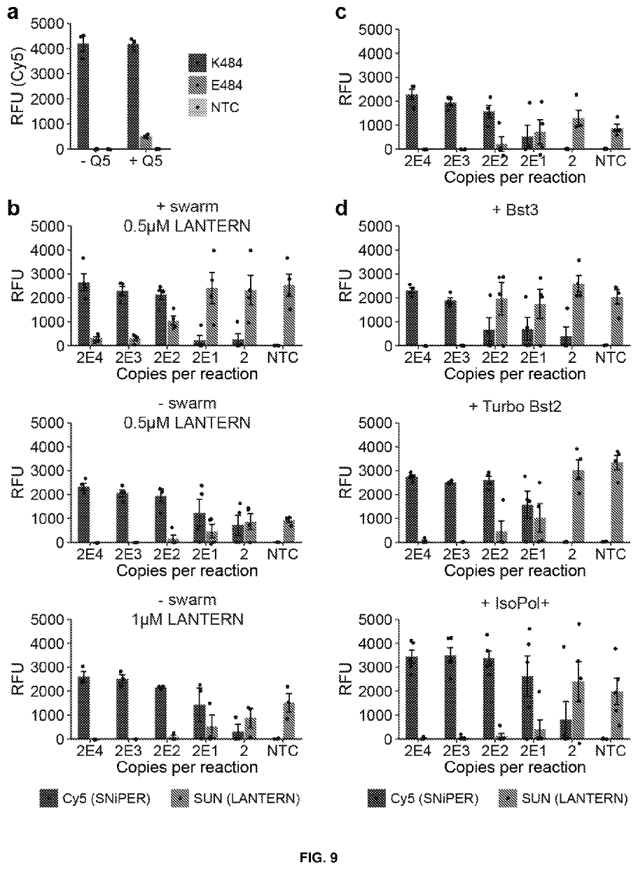

For a diagnostic assay to be deployed, it should contain an internal control to ensure that a negative test result is not due to an insufficient amount of sample added, for example, an internal control may include detecting human ACTB. However, a high-fidelity DNA polymerase is required. Hence, it was checked whether the addition of such an enzyme would disrupt the functioning of the SNIPER probe and detection method described herein, however, it was found that the Q5 high-fidelity polymerase could barely cleave mismatched LNA nucleotides (FIG. 9a). Hence, LANTERN and SNIPER probes are compatible in the same reaction mix.

A 2-plex reaction was run with a SUN-conjugated LANTERN probe against ACTB and a 21nt Cy5-conjugated SNIPER probe against the viral S-gene. Variable amounts of in vitro transcribed mutant (K484) RNA were spiked into 0.25 ng of PC9 RNA to simulate purified patient samples (FIG. 9b). Each reaction mix was also supplemented with extra Bst2 enzyme as before. Moreover, to reduce competition for reagents between the ACTB and S-gene LAMP primers, the concentration of ACTB primers was lowered to 0.2X. However, in the initial 2-plex reactions, it was observed that the fluorescence signal in the Cy5 channel and the sensitivity of the SNIPER probe were compromised. Hence, to further ease the competition, both swarm primers were removed from the ACTB set of LAMP primers. Assay sensitivity was improved, but fluorescence in the SUN channel fell by over 2-fold. Moreover, increasing the concentration of ACTB LANTERN probe from 0.5 μM to 1 μM did not restore the original level of signal in the SUN channel. Nevertheless, it was confirmed that the extra Bst2 enzyme was necessary because replacing it with extra reverse transcriptase instead degraded the performance of the 2-plex assay (FIG. 9c).

Since fluorescence is generated only when the quencher is cleaved off the SNIPER probe by an incoming DNA polymerase, a possible limiting factor is that the Bst2 enzyme-in-use is not sufficiently processive. Hence, it was asked if addition of other engineered Bst polymerases could enhance the fluorescence signal and improve the sensitivity of the assay. To this end, three different enzymes were evaluated, namely Bst3, Turbo Bst2, and IsoPol+ (FIG. 9d). All three Bst polymerases boosted the fluorescence signal for ACTB in the SUN channel. However, they exhibited variable effects on the fluorescence signal in the Cy5 channel. Specifically, while addition of Bst3 degraded the assay sensitivity for the virus, addition of IsoPol+clearly enhanced the output signal instead. Hence, IsoPol+ was included in subsequent multiplexed assays containing a human internal control.

### Example 4: Direct application of SNIPER assay on unpurified samples

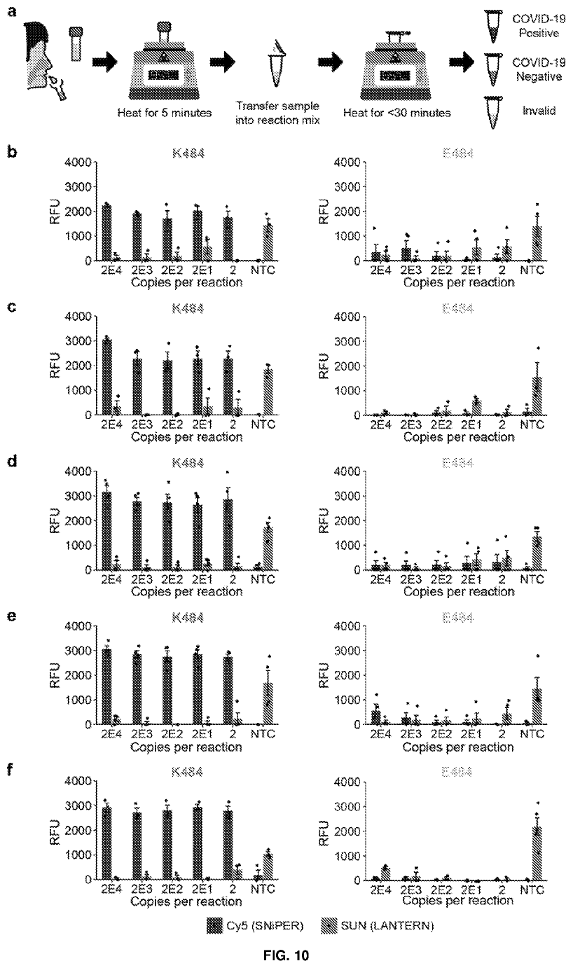

For it to be more widely deployable, a POC test should ideally work on patient samples directly without the need for RNA extraction. Hence, the sensitivity of the method described herein was evaluated using saliva samples spiked with variable copies of lentivirus that expressed either the K484 or E484 S-gene fragment. The contrived specimens were treated with Proteinase K for 1 minute prior to heating at 95° C. for 5 minutes, which would inactivate the proteinase and promote lysis of the viral particles (FIG. 10a). The reaction mix was also supplemented with extra Turbo Bst2 (FIG. 10b) or IsoPol+ (FIG. 10c). In both cases, reasonably strong fluorescence signal was obtained for the K484 viral target even at 2 copies per reaction, while the signal for the E484 template remained at near-background levels. Consistent with earlier data, the fluorescence obtained with IsoPol+ was higher than those obtained with Turbo Bst2.

Next, strategies to boost the signal for the intended target was explored. First, 50 mM EDTA was added, which chelates metal ions and helps to protect RNA from degradation, into the lysis solution and found that it improved the fluorescence signal for the K484 lentivirus (FIG. 10d). Second, a commercially available ZeroPrep Saliva Collection Kit was tested, which contained proteinase K and SDS detergent in the buffer, and observed a better signal for the perfectly matched target as well (FIG. 10e). Nevertheless, despite the promising results, some low fluorescence signals for the mismatched E484 template was observed, which was most likely due to residual Q5 proofreading activity. Hence, to completely inhibit this activity on the SNIPER probe, an extra LNA residue at the terminal 3′ end of the probe was utilised. A re-test of the contrived specimens processed with the ZeroPrep kit showed that the low but undesired fluorescence signals for the mismatched template had essentially been abolished by the addition of another LNA (FIG. 10f). Taken together, the results demonstrate that the method described herein can unambiguously discriminate between K484 and E484 regardless of viral titres.

### Example 5: A 3-plex assay to identify E484 wildtype virus and K484 mutant virus

In multiplex assays so far, fluorescence was emitted only in the presence of human ACTB or viral S-gene with a K484 mutation. However, there are many viral strains that do not harbour this mutation. The 2-plex setup would not be able to distinguish between the presence of all these other strains, including the original wildtype virus with E484 in the spike, and the complete absence of SARS-COV-2 (in the case of a healthy individual).

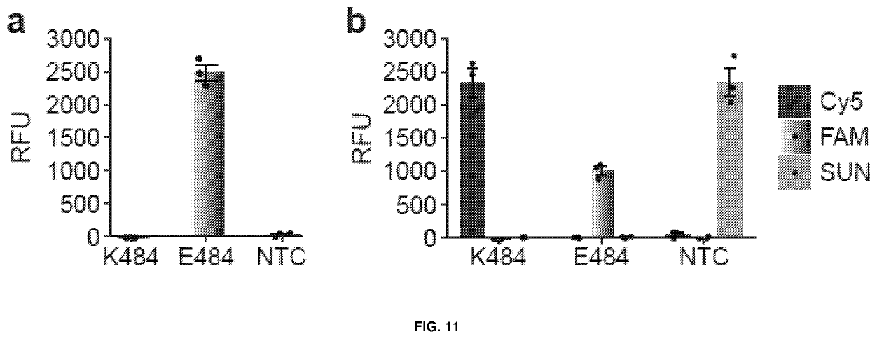

To address the issue, a 3-plex assay was sought to be developed whereby besides the internal control, both E484 and K484 in the spike could be specifically identified with different fluorophores. For this purpose, a FAM-conjugated probe was developed that was perfectly matched with the E484 template. Expectedly, in 1-plex reactions with in vitro transcribed RNA, the probe gave a clear fluorescence signal only in the presence of the intended E484 target (FIG. 11a).

RT-LAMP reactions were then set-up containing three fluorescent probes together:
the FAM-conjugated SNIPER probe against wildtype viral S-gene (E484);
a Cy5-conjugated SNIPER probe against mutated viral S-gene (K484); and
a HEX-conjugated LANTERN probe against human ACTB.

Each reaction mix was supplemented with IsoPol+. As template, PC9 RNA spiked with synthetic wildtype or mutant SARS-COV-2 RNA was used. Fluorescence in the FAM and Cy5 channels was observed only for the E484 and K484 templates respectively (FIG. 11b). Moreover, human ACTB was amplified only in the absence of viral RNA by design as only 0.2λ ACTB LAMP primers was used. Hence, the results indicate that a 3-plex method described herein can specifically identify wildtype or K484 mutant virus in human samples.

### Example 6: SNIPER probes to detect different SARS-COV-2 variants

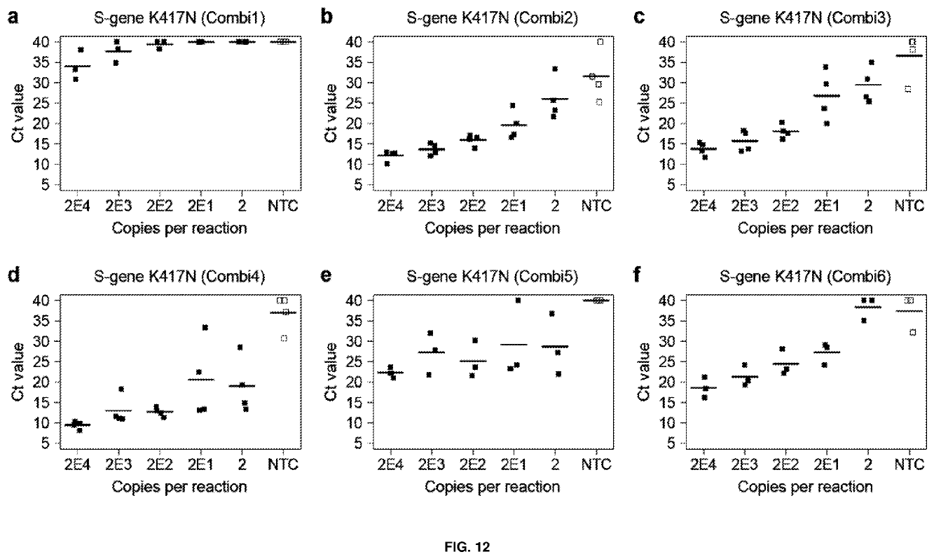

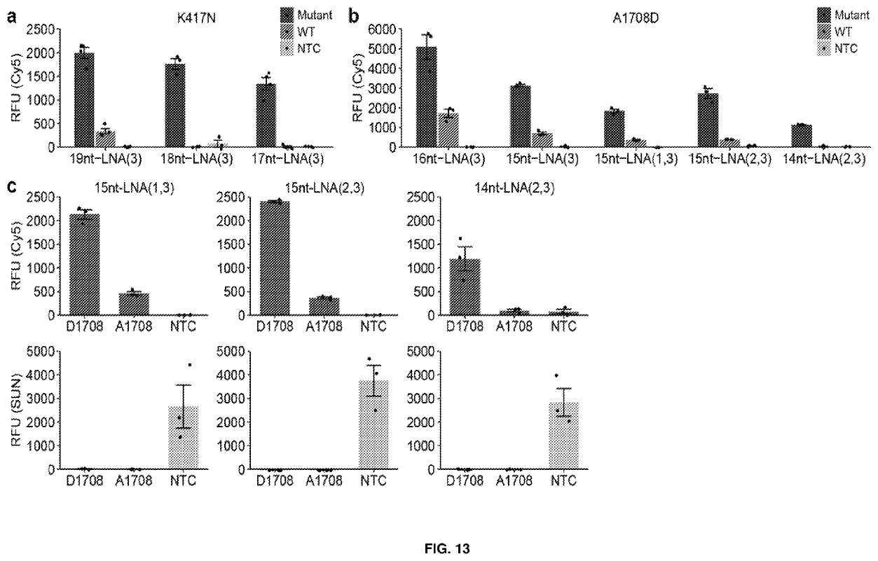

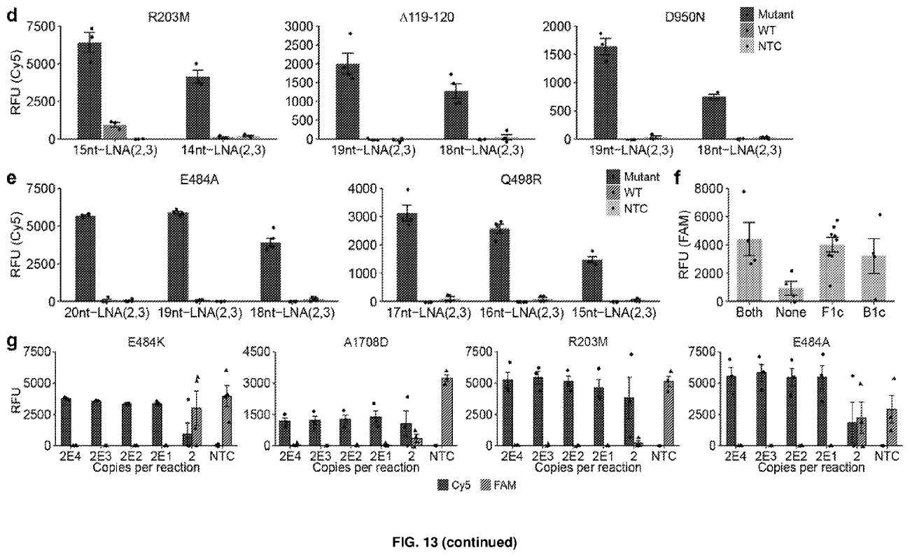

Encouraged by results for the E484K mutation, probes for other SARS-COV-2 mutations were designed. First, the K417N mutation was focused on, which was present not only in the Beta variant like E484K but also in the Delta Plus variant as well. After screening for the optimal LAMP primer set (FIG. 12), probes of three different lengths were tested. Each probe would bind with perfect complementarity to the variant sequence, with the altered nucleotide located at the −2 position and an LNA residue located at the −3 position. While the 19nt probe gave a 5.9-fold higher signal for the variant than for the wildtype template, the mutant-to-wildtype signal ratio was greater than 10 for both the 18nt and 17nt probes, providing clear differentiation between Beta/Delta Plus and other viral variants (FIG. 13a, 20a). Table 3 below shows the melting temperatures of SNIPER probes at different lengths targeting the K417N (SARS-COV2 S-gene) mutations.

TABLE 3

| | Sequence | SEQ | | | |
|---|---|---|---|---|---|
| | (underlined bases | ID | | | |
| Name | denote LNA) | NO | Length | Tm-PM | Tm-MM |
| 19-LNAnt3 | CCAGGGCAAACTGGAAATA | 130 | 19 | 64.9 | 62.6 |
| 18-LNAnt3 | CAGGGCAAACTGGAAATA | 131 | 18 | 62.5 | 59.6 |
| 17-LNAnt3 | AGGGCAAACTGGAAATA | 132 | 17 | 61.3 | 57.8 |

Next, several SNIPER probes were evaluated for the A1708D site present in the Alpha variant. Earlier, the LANTERN probe for this locus yielded a mutant-to-wildtype signal ratio of only 3.9-fold (FIG. 1b and FIG. 5a). Here, two LNA (3) probes of different lengths were tested, but still observed appreciable fluorescence signal for the wildtype template (FIG. 13b). Since LNA residues may confer greater mismatch discrimination than canonical nucleotides, an extra LNA at either the −1 or −2 position was introduced. RT-LAMP assays yielded a mutant-to-wildtype signal ratio of 4.5-fold, 5.0-fold, and 6.9-fold for LNA (3), LNA (1,3), and LNA (2,3) respectively. Further reduction of LNA (2,3) probe length from 15nt to 14nt increased the ratio to over 10-fold. Hence, two LNA residues at the −2 and −3 positions exhibited the strongest SNP discrimination capability. Additionally, it was found that LNA (2,3) probes exhibited similar wildtype fluorescence signal in the presence of Q5 high-fidelity DNA polymerase (FIG. 13c), indicating that they were not cleavable by Q5 like LNA (1,3) and thus were compatible with LANTERN probes as well. Hence, moving forward, LNA residues at both the −2 and −3 positions were included for all subsequent SNIPER probes.

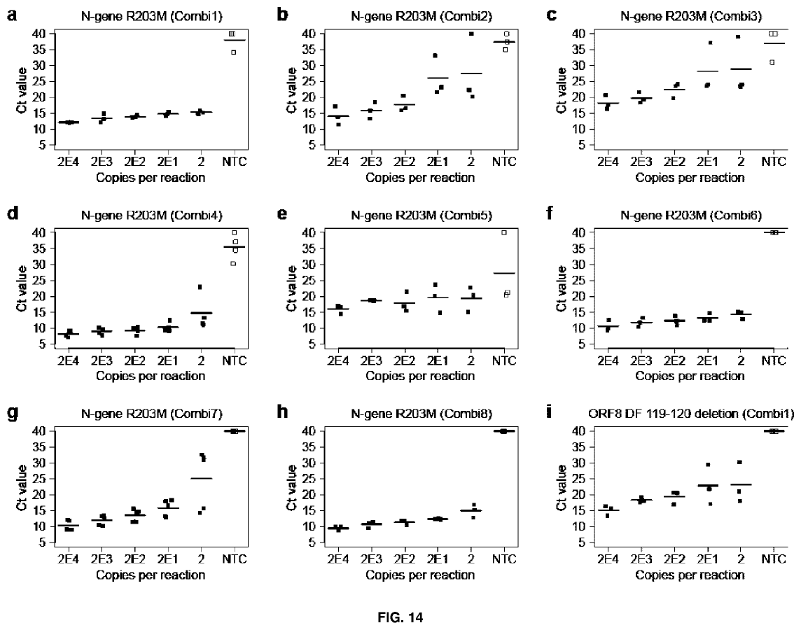

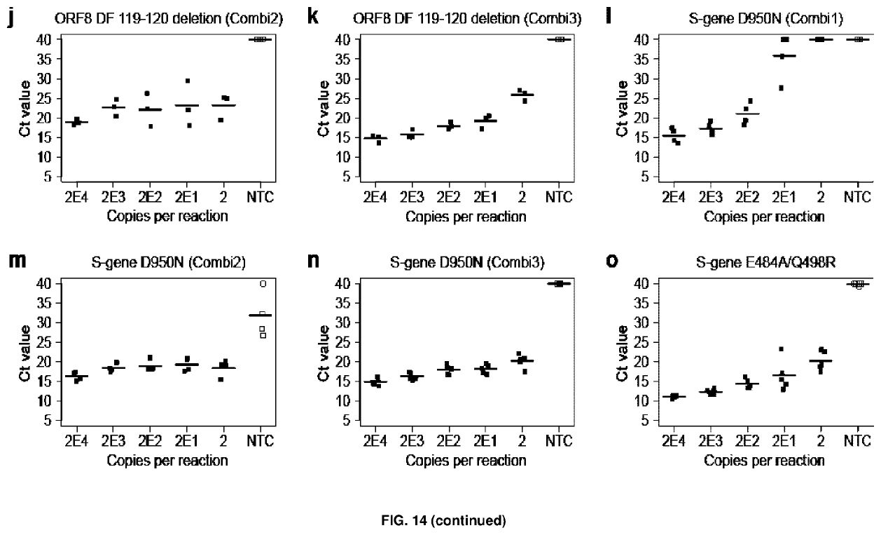

Recent COVID-19 infection waves were driven by the Delta variant and then the Omicron variant. Therefore, after screening for the best LAMP primer sets (FIG. 14), SNIPER probes of different lengths for three Delta mutations were evaluated (FIG. 13d) and two Omicron mutations (FIG. 13e). Most tested probes showed high fluorescence signal for the target mutation and negligible background levels of fluorescence for the wildtype template, highlighting the versatility of our SNIPER method.

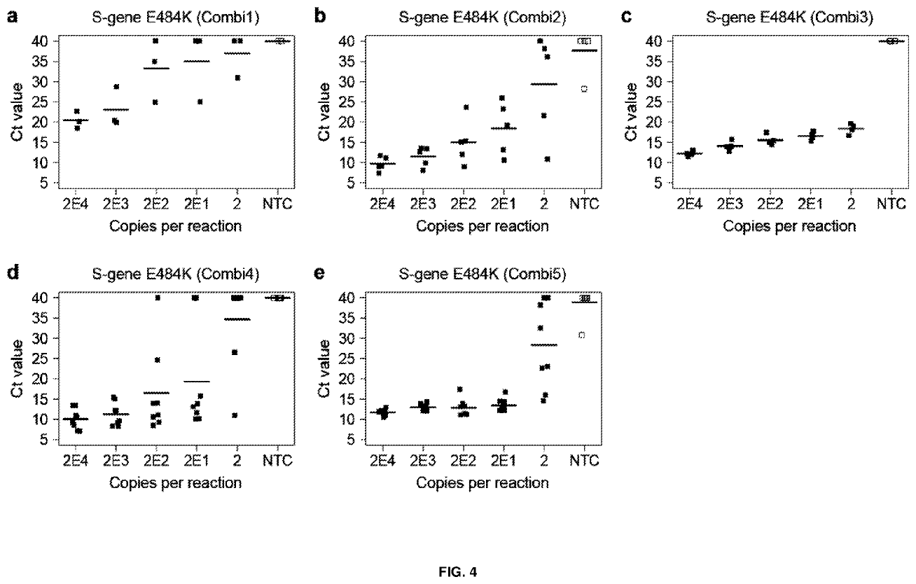

Subsequently, it was sought to incorporate a human internal control into the assays for the different SARS-COV-2 variants. Here, it was aimed to employ a SNIPER probe for ACTB to obviate the need for the Q5 polymerase, thereby lowering consumables cost for the user. A FAM-conjugated probe was tested in the absence or presence of one or both swarm primers using 0.25 ng PC9 RNA as template (FIG. 13f). From the results, it was chosen to proceed with only one swarm primer, F1c, for the internal control, since it led to successful amplification of the human target in all replicates and our earlier experiments showed that viral sensitivity was affected by the presence of two ACTB swarm primers (FIG. 4b). A 2-plex assays for the Beta, Alpha, Delta, and Omicron variants was evaluated using only SNIPER probes. For all selected sites, the analytical LoD for the coronavirus was 20 copies per reaction (FIG. 13g), which was close to that of qRT-PCR assays. Moreover, human ACTB was amplified in all NTC reactions, providing assurance that a negative test was not due to inadequate sample loading. Collectively, this data demonstrates that the SNIPER probe described herein can be used to sensitively detect any specific SARS-COV-2 variant.

### Example 7: Addition of a third probe for variant-independent detection

In a 2-plex assay, amplification of the human internal control was largely outcompeted by the viral template because the ACTB LAMP primers were loaded at only 0.2λ concentration (FIG. 10). Consequently, in the presence of a wildtype virus, minimal fluorescence signal was observed in both channels since the SNIPER probe would be unable to detect the mismatched wildtype amplicon. The fluorescence signal for human ACTB was only detectable primarily in samples without any viral template. Although one may interpret an absence of signal in both channels to be a positive test result for a virus lacking the target mutation, the no-signal outcome may also be caused by insufficient sample loading or spoilage of reagents.

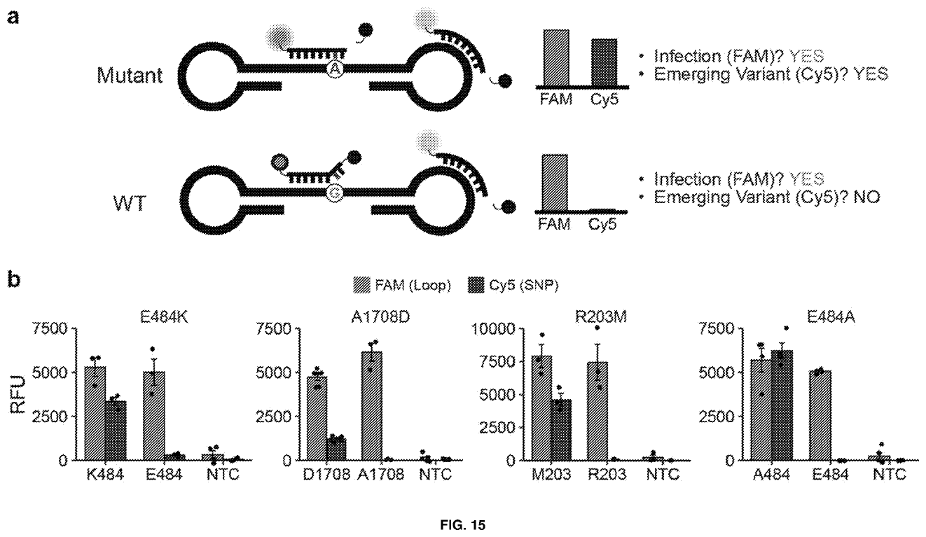

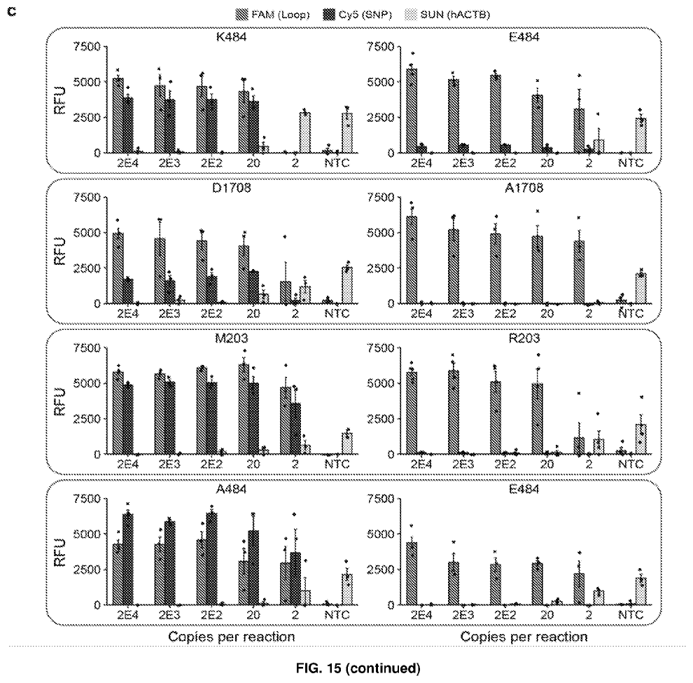

To eliminate the ambiguity in result interpretation, we sought to incorporate an extra probe for the detection of any viral strain, providing information on the infection status of an individual. As the SNIPER probes for all chosen mutations targeted the stem region of each LAMP product, FAM-conjugated probes were designed against the loop region of the dumbbell amplicon instead (FIG. 15a). Here, binding sites were selected that were highly conserved across over 99% of the genomes deposited in GISAID. Hence, when a fluorescence signal is observed in both the Cy5 and FAM channels, the individual is infected with a variant. However, when fluorescence is observed in only the FAM channel but not the Cy5 channel, the individual is infected with a virus lacking the target SNP.

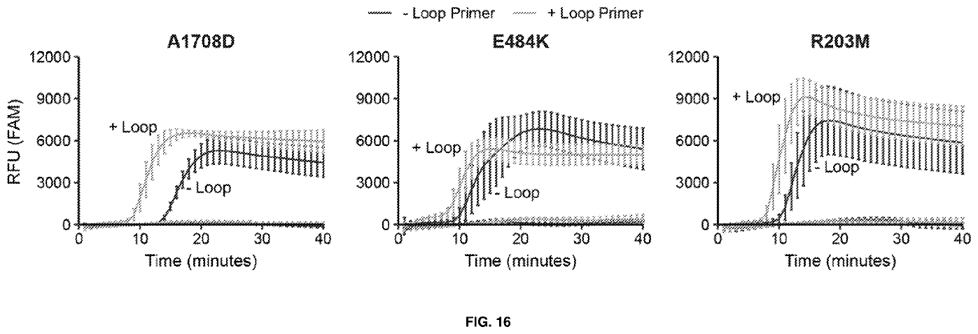

FAM-conjugated loop probes were designed for Beta, Alpha, Delta, and Omicron assays. The same loop probe can be used for E484K and E484A. Since LAMP loop primers (without fluorophore and quencher) may interfere with the SNIPER loop probe, each new probe was first tested in reactions with or without the corresponding loop primer (FIG. 16). Unexpectedly, reactions with both the primer and the probe showed faster amplification than those with only the probe, while the fluorescence intensities were similar. This indicates that the SNIPER probe cannot fully replace the original loop primer, possibly because the Bst polymerase must cleave off the quencher at the 3′ end of the probe before extension can occur. Moving forward, all loop primers were included in the assays.

Next, each FAM-conjugated loop probe was evaluated with the corresponding Cy5-conjugated SNP probe using mutant or wildtype synthetic RNA as template. In 1-plex assays, for all the four tested sites, fluorescence was readily detected in both channels when mutant viral RNA carrying the SNP was added but was observed only in the FAM channel but not the Cy5 channel when wildtype RNA was used (FIG. 15b). A hACTB probe and primers were incorporated at 0.2λ concentration into the assay as an internal control and evaluated the sensitivity of this 2-plex, 3-probe setup by spiking the viral template into human PC9 RNA (FIG. 15c). Remarkably, the SNIPER assay still achieved an analytical LoD of 20 copies per reaction as before for both mutant and wildtype templates. Importantly, for all the four sites tested, the mutant-to-wildtype signal ratio was higher than 10-fold regardless of the template copy number. It was also observed that the green and red fluorescence signals for the virus were starting to plateau at around 20 minutes. However, amplification of the human internal control was slower as the ACTB primers were diluted to 0.2λ concentration. Therefore, 30 minutes was maintained as the final timepoint. Collectively, the results demonstrate that the expanded assay can inform whether an individual is healthy, infected with a specific SARS-CoV-2 variant, or infected with a virus lacking the target SNP.

### Example 8: Evaluation of direct lyophilised 3-probe assays

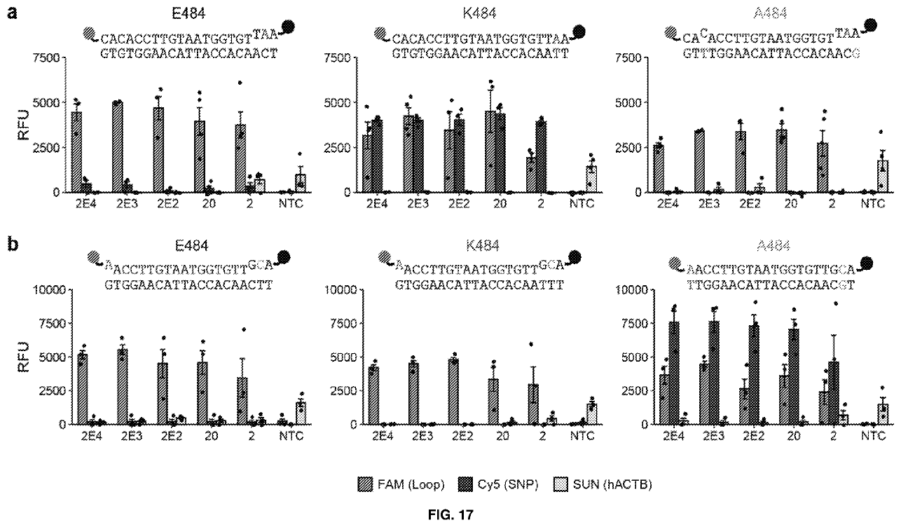

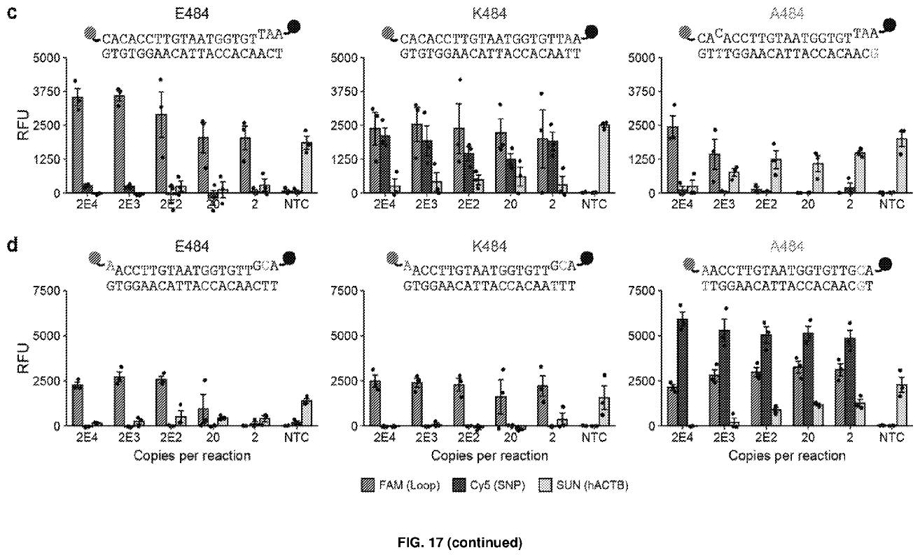

To facilitate point-of-care testing, diagnostic assays should work on unpurified patient samples. Hence, a 3-probe SNIPER assay was evaluated using saliva samples spiked with variable copies of lentivirus that expressed E484 (wildtype), K484 (Beta), or A484 (Omicron) S-gene fragment. The contrived specimens were treated with Proteinase K and EDTA, before heating at 95° C. for 5 minutes to inactivate the proteinase and promote lysis of the virus (FIG. 10a). Both K484 (FIG. 17a) and A484 (FIG. 17b) SNIPER probes were tested against the three viruses. Expectedly, each SNP probe fluoresced only when the fully matching template was added. The analytical LoD achieved was 20 copies per reaction, with the mutant-to-wildtype signal ratio maintained at greater than 10-fold for all positive outcomes. Moreover, the loop probe detected every virus regardless of its SNP status.

### Example 9: General applicability of SNIPER probes

The general utility of the SNIPER probes is demonstrated in Example 6 above in the identification of genetic variants beyond the E484K mutation in the S-gene for SARS-COV-2.

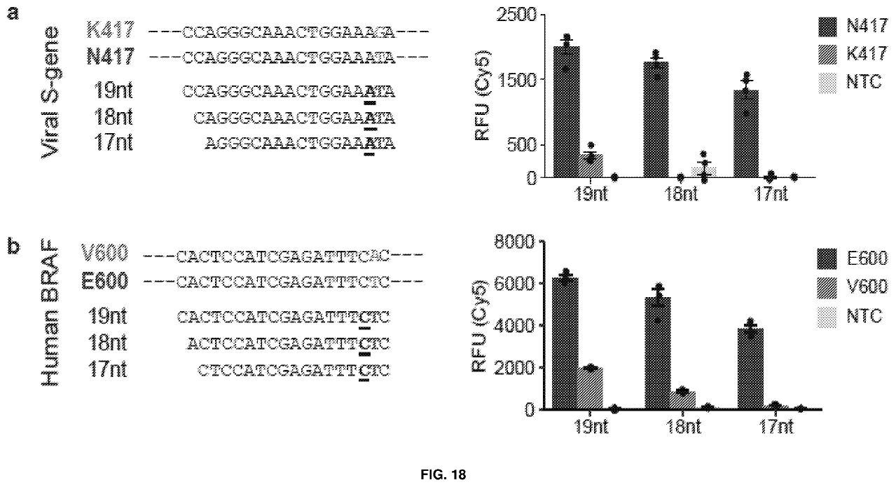

For example, probes to detect another K417N mutation in the spike were designed, which is present in the Beta and Delta Plus variants. Each probe was perfectly matched with the N417-encoding sequence and contained an internal LNA residue at the −3 position. N417 probes were evaluated of three different lengths (FIG. 18a). Overall, it was found that in the presence of the intended target, all three SNIPER probes produced fluorescence that was clearly above background, although the signal was slightly reduced for shorter probes. However, the longest 19nt probe, but not the other two shorter oligonucleotides, also gave a low but appreciable signal in the presence of a mismatched template.

As a different application, it was asked whether the SNIPER probes could be used to detect cancer mutations. To this end, probes against the V600E mutation in the human BRAF gene were designed and tested. This mutation is common in multiple neoplasms, including melanoma and colorectal cancer. Again, E600 probes of three different lengths were evaluated, with each containing an internal LNA residue at the −3 position (FIG. 18b). While all three probes gave strong fluorescence for the perfectly matched E600 template, the longer probes also yielded some signal in the presence of the mismatched wildtype template. Only the shortest probe, of length 17nt, gave near-background fluorescence readings with the V600 template, resulting in a >10 mutant-to-wildtype signal ratio. Collectively, the results indicate that the melting temperature of the probes can be tuned by adjusting the oligonucleotide length, which then allows us to eliminate or at least minimize unwanted signal from mismatched substrates. Table 4 below shows the melting temperatures of SNIPER probes at different lengths targeting the V600E (human BRAF) mutation.

TABLE 4

| | Sequence | SEQ | | | |
|---|---|---|---|---|---|
| | (underlined bases | ID | | | |
| Name | denote LNA) | NO | Length | Tm-PM | Tm-MM |
| 19-LNAnt3 | CACTCCATCGAGATTTCTC | 133 | 19 | 67 | 62 |
| 18-LNAnt3 | ACTCCATCGAGATTTCTC | 134 | 18 | 65 | 59 |
| 17-LNAnt3 | CTCCATCGAGATTTCTC | 135 | 17 | 63 | 56 |

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objective and obtain the ends and advantages mentioned, as well as those inherent therein. Further, it will be readily apparent to one skilled in the art that varying substitutions and modifications may be made to the invention disclosed herein without departing from the scope and spirit of the invention. The methods, kits and uses described herein are presently representative of preferred embodiments and are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

1 Tian, F. et al. N501Y mutation of spike protein in SARS-COV-2 strengthens its binding to receptor ACE2. Elife 10, doi: 10.7554/eLife.69091 (2021).
2 Wang, P. et al. Antibody resistance of SARS-COV-2 variants B. 1.351 and B.1.1.7. Nature 593, 130-135, doi: 10.1038/s41586-021-03398-2 (2021).
3 Greaney, A. J. et al. Comprehensive mapping of mutations in the SARS-COV-2 receptor-binding domain that affect recognition by polyclonal human plasma antibodies. Cell Host Microbe 29, 463-476 e466, doi: 10.1016/j.chom.2021.02.003 (2021).
4 Wang, Z. et al. mRNA vaccine-elicited antibodies to SARS-COV-2 and circulating variants. Nature 592, 616-622, doi: 10.1038/s41586-021-03324-6 (2021).
5 Chen, R. E. et al. Resistance of SARS-COV-2 variants to neutralization by monoclonal and serum-derived polyclonal antibodies. Nat Med 27, 717-726, doi: 10.1038/s41591-021-01294-w (2021).
6 Liu, Z. et al. Identification of SARS-COV-2 spike mutations that attenuate monoclonal and serum antibody neutralization. Cell Host Microbe 29, 477-488 e474, doi: 10.1016/j.chom.2021.01.014 (2021).
7 Lee, W. L. et al. Quantitative SARS-COV-2 Alpha Variant B.1.1.7 Tracking in Wastewater by Allele-Specific RT-qPCR. Environmental Science & Technology Letters 8, 675-682, doi: 10.1021/acs.estlett.1c00375 (2021).
8 Vega-Magana, N. et al. RT-qPCR Assays for Rapid Detection of the N501Y, 69-70del, K417N, and E484K SARS-COV-2 Mutations: A Screening Strategy to Identify Variants With Clinical Impact. Front Cell Infect Microbiol 11, 672562, doi: 10.3389/fcimb.2021.672562 (2021).
9 Vogels, C. B. F. et al. Multiplex qPCR discriminates variants of concern to enhance global surveillance of SARS-COV-2. PLOS Biol 19, e3001236, doi: 10.1371/journal.pbio.3001236 (2021).
10 Yaniv, K. et al. Direct RT-qPCR assay for SARS-COV-2 variants of concern (Alpha, B.1.1.7 and Beta, B.1.351) and detection quantification in wastewater. Environ Res 201, 111653, doi: 10.1016/j.envres.2021.111653 (2021).
11 de Puig, H. et al. Minimally instrumented SHERLOCK (miSHERLOCK) for CRISPR-based point-of-care diagnosis of SARS-COV-2 and emerging variants. Sci Adv 7, doi: 10.1126/sciadv.abh2944 (2021).
12. Dao Thi, V. L. et al. A colorimetric RT-LAMP assay and LAMP-sequencing for detecting SARS-COV-2 RNA in clinical samples. Sci Transl Med 12, doi: 10.1126/scitranslmed.abc7075 (2020).
13. Ganguli, A. et al. Rapid isothermal amplification and portable detection system for SARS-COV-2. Proc Natl Acad Sci USA 117, 22727-22735, doi: 10.1073/pnas.2014739117 (2020).
14. Thompson, D. & Lei, Y. Mini review: Recent progress in RT-LAMP enabled COVID-19 detection. Sensors and Actuators Reports 2, doi: 10.1016/j.snr.2020.100017 (2020).
15. Badolo, A. et al. Detection of G119S ace-1 (R) mutation in field-collected Anopheles gambiae mosquitoes using allele-specific loop-mediated isothermal amplification (AS-LAMP) method. Malar J 14, 477, doi: 10.1186/s12936-015-0968-9 (2015).
16. Badolo, A. et al. Development of an allele-specific, loop-mediated, isothermal amplification method (AS-LAMP) to detect the L1014F kdr-w mutation in Anopheles gambiae s. I. Malar J 11, 227, doi: 10.1186/1475-2875-11-227 (2012).
17. Malpartida-Cardenas, K. et al. Allele-Specific Isothermal Amplification Method Using Unmodified Self-Stabilizing Competitive Primers. Anal Chem 90, 11972-11980, doi: 10.1021/acs.analchem.8b02416 (2018).
18. Itonaga, M. et al. Novel Methodology for Rapid Detection of KRAS Mutation Using PNA-LNA Mediated Loop-Mediated Isothermal Amplification. PLOS One 11, e0151654, doi: 10.1371/journal.pone.0151654 (2016).
19. Du, W. F. et al. Single-step, high-specificity detection of single nucleotide mutation by primer-activatable (PA-LAMP). Anal loop-mediated isothermal amplification Chim Acta 1050, 132-138, doi: 10.1016/j.aca.2018.10.068 (2019).
20. Shen, H. et al. A Sensitive, Highly Specific Novel Isothermal Amplification Method Based on Single-Nucleotide Polymorphism for the Rapid Detection of Salmonella Pullorum. Front Microbiol 11, 560791, doi: 10.3389/fmicb.2020.560791 (2020).
21. Ding, X., Yin, K., Chen, J., Wang, K. & Liu, C. A ribonuclease-dependent cleavable beacon primer triggering DNA amplification for single nucleotide mutation detection with ultrahigh sensitivity and selectivity. Chem Commun (Camb) 55, 12623-12626, doi: 10.1039/c9cc06296c (2019).
22. Higgins, O. & Smith, T. J. Loop-Primer Endonuclease Cleavage-Loop-Mediated Isothermal Amplification Technology for Multiplex Pathogen Detection and Single-Nucleotide Polymorphism Identification. J Mol Diagn 22, 640-651, doi: 10.1016/j.jmoldx.2020.02.002 (2020).
23. Ayukawa, Y., Hanyuda, S., Fujita, N., Komatsu, K. & Arie, T. Novel loop-mediated isothermal amplification (LAMP) assay with a universal QProbe can detect SNPs determining races in plant pathogenic fungi. Sci Rep 7, 4253, doi: 10.1038/s41598-017-04084-y (2017).
24. Jiang, Y. S. et al. Robust strand exchange reactions for the sequence-specific, real-time detection of nucleic acid amplicons. Anal Chem 87, 3314-3320, doi: 10.1021/ac504387c (2015).
25. Liu, W. et al. Establishment of an accurate and fast detection method using molecular beacons in loop-mediated isothermal amplification assay. Sci Rep 7, 40125, doi: 10.1038/srep40125 (2017).
26 Nyan, D. C. & Swinson, K. L. A novel multiplex isothermal amplification method for rapid detection and identification of viruses. Sci Rep 5, 17925, doi: 10.1038/srep17925 (2015).
27. Varona, M. & Anderson, J. L. Visual Detection of Single-Nucleotide Polymorphisms Using Molecular Beacon Loop-Mediated Isothermal Amplification with Centrifuge-Free DNA Extraction. Anal Chem 91, 6991-6995, doi: 10.1021/acs.analchem.9b01762 (2019).
28. Ding, X. et al. Cleavable hairpin beacon-enhanced fluorescence detection of nucleic acid isothermal amplification and smartphone-based readout. Sci Rep 10, 18819, doi: 10.1038/s41598-020-75795-y (2020).
29. Arizti-Sanz, J. et al. Streamlined inactivation, amplification, and Cas13-based detection of SARS-CoV-2. Nat Commun 11, 5921, doi: 10.1038/s41467-020-19097-x (2020).
30. Kean Hean Ooi et al. A Sensitive and Specific Fluorescent RT-LAMP Assay for SARS-COV-2 Detection in Clinical Samples. ACS Synth Biol 11 (1): 448-463, doi: 10.1021/acssynbio. 1c00538. (2022).
31. Mary Hongying Cheng et al. Impact of new variants on SARS-COV-2 infectivity and neutralization: A molecular assessment of the alterations in the spike-host protein interactions. iScience. 2022 Mar. 18; 25 (3): 103939. Published online 2022 Feb. 17. doi: 10.1016/j.isci.2022.103939
32. McTigue et al. Sequence-Dependent Thermodynamic Parameters for Locked Nucleic Acid (LNA)-DNA Duplex Formation. ACS Biochemistry 2004, 43, 18, 5388-5405.

1. A method for determining the presence, or quantity, of a point mutation, preferably a single nucleotide polymorphism (SNP), in a target nucleic acid molecule in a sample using loop-mediated isothermal amplification (LAMP), the method comprising:
(a) combining a LAMP reaction mixture, DNA polymerase with 5′→3′ polymerase activity, and a detection probe with the sample (suspected of containing the target nucleic acid molecule),
wherein the LAMP reaction mixture comprises a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule,
wherein the detection probe recognises a probe binding site within target amplicons,
wherein the detection probe is a single-stranded probe comprising:
a nucleotide sequence fully complementary to a nucleotide sequence of the probe binding site comprising the point mutation;
a nucleotide complementary to the point mutation at the penultimate base relative to the 3′ end of the detection probe; and
a locked nucleic acid (LNA) or peptide nucleic acid (PNA) residue at the first, second, third and/or fourth, preferably third, or second and third, or first and third, or third and fourth nucleotide position relative to the 3′ end of the detection probe,
wherein the detection probe comprises a quencher-fluorophore pair at opposite ends of the probe at a distance that allow the quencher to quench the fluorophore signal, wherein the detection probe can hybridize to said target amplicons under LAMP assay conditions and form a double-stranded probe: target complex,
(b) amplifying the target nucleic acid molecule by LAMP under suitable assay conditions that allow:
i. generation of the target amplicons;
ii. hybridization of the detection probe to the target amplicons to form the probe: target complex; and
iii. cleavage of the detection probe by the DNA polymerase to release the quencher or fluorophore; and
(c) detecting and optionally quantifying the released quencher or fluorophore to determine the presence or quantity of the point mutation in the target nucleic acid molecule in the sample.

2. The method of claim 1, wherein the DNA polymerase with 5′→3′ polymerase activity is a Bst polymerase selected from Bst3 polymerase, Bst2 polymerase and IsoPol+.

3. The method of claim 1, wherein the primer set further comprises two swarm primers.

4. The method of claim 1, wherein the primers are designed to amplify loci in the target nucleic acid molecule comprising the point mutation relative to a reference wild-type nucleotide sequence, wherein the nucleotide sequence of the probe binding site comprises the point mutation.

5. (canceled)

6. The method of claim 1, wherein the LNA or PNA residue is positioned at the third nucleotide position relative to the 3′ end of the detection probe.

7. The method of claim 1, wherein the LNA or PNA residue is positioned at the first and third or second and third nucleotide position relative to the 3′ end of the detection probe.

8. The method of claim 1, wherein the probe binding site is different from and non-overlapping with any one of the primer binding sites.

9. The method of claim 1, wherein the detection probe is 14-23 nucleotide bases in length, preferably 14-21 nucleotide bases in length.

10. The method of claim 1, wherein the detection probe comprises a phosphorothioate bond at the 3′-end.

11. The method of claim 1, wherein the detection probe comprises at least one additional modified nucleotide residue to either increase the detection probes melting temperature (Tm) or binding affinity.

12. The method of 1, wherein the fully complementary double-stranded probe: target complex has a higher melting temperature (Tm) in comparison to a non-fully complementary double-stranded probe: target complex in which the target nucleic acid comprises at least one mismatched nucleotide.

13. The method of claim 12, wherein the melting temperature (Tm) of the fully complementary double-stranded probe: target complex is higher by about 1.5° C. or greater in comparison to the non-fully complementary double-stranded probe: target complex.

14. The method of claim 1, wherein the fluorophore is attached to the 5′ end of the detection probe and the quencher is attached to the 3′ end of the detection probe.

15. The method of claim 1, wherein the method is a multiplexing method and is for determining the presence, or optionally quantity, of a point mutation in two or more target nucleic acid molecules in the sample, wherein the method uses one or more primer sets and one or more detection probes for each target nucleic acid molecule or for multiple related target nucleic acid molecules.

16. The method of claim 1, wherein the point mutation is a biomarker for a disease or condition in a subject, optionally cancer.

17. The method of claim 1, wherein the target nucleic acid molecule is a nucleic acid of a pathogen, optionally a human pathogen, preferably a bacterial, fungal, parasite or viral nucleic acid molecule or a cDNA reverse transcript of a bacterial, fungal, parasite or viral RNA.

18. The method of claim 17, wherein the pathogen is a coronavirus, influenza virus, paramyxovirus or enterovirus.

19. The method of claim 18, wherein the target nucleic acid molecule is a nucleic acid of SARS-COV-2 virus, optionally variants of the SARS-COV-2 virus selected from alpha, beta, gamma and delta variants, optionally wherein the nucleic acid of SARS-CoV-2 virus is an S-gene comprising a point mutation relative to a reference wild-type S-gene nucleotide sequence.

20. (canceled)

21. The method of claim 1, wherein the sample is not subjected to a purification step prior to step (a).

22. (canceled)

23. A kit for determining the presence, or quantity, of a point mutation, preferably single nucleotide polymorphism (SNP), in a target nucleic acid molecule in a sample using loop mediated isothermal amplification (LAMP), the kit comprising:
a LAMP reaction mixture comprises a LAMP primer set of 4 to 6 primers, comprising two inner primers (FIP and BIP), two outer primers (F3 and B3), and optionally one or two loop primers (LF and/or LB), wherein each primer recognises a distinct primer binding site within the target nucleic acid molecule;
a DNA polymerase with 5′→3′ polymerase activity; and
a detection probe that recognises a probe binding site within target amplicons, wherein the detection probe is a single-stranded probe comprising:
a nucleotide sequence fully complementary to a nucleotide sequence of the probe binding site comprising the point mutation;
a nucleotide complementary to the point mutation at the penultimate base relative to the 3′ end of the detection probe; and
a locked nucleic acid (LNA) or peptide nucleic acid (PNA) residue at the first, second, third and/or fourth, preferably third, or second and third, or first and third, or third and fourth nucleotide position relative to the 3′ end of the detection probe,
wherein the detection probe comprises a quencher-fluorophore pair at opposite ends of the probe at a distance that allows the quencher to quench the fluorophore signal, wherein the detection probe can hybridize to said target amplicons under LAMP assay conditions and form a double-stranded probe: target complex.
